(19) United States
(12) Patent Application Publication
(10) Pub. No.: US 2014/0051586 A1
(43) Pub. Date: Feb. 20, 2014

(54) HIGH-THROUGHPUT METHODS TO PRODUCE, VALIDATE AND CHARACTERIZE MMABS

(71) Applicant: Ignacio Pino, Mayaguez, PR (US)

(72) Inventor: Ignacio Pino, Mayaguez, PR (US)

(21) Appl. No.: 13/981,308

(22) PCT Filed: Jan. 25, 2012
(86) PCT No.: PCT/US12/22608

§ 371 Date: Nov. 4, 2013

Related U.S. Application Data
(60) Provisional application No. 61/436,121, filed on Jan. 25, 2011.

(51) Int. Cl. G01N 33/543

(52) U.S. Cl. CPC G01N 33/54306
USPC 506/2; 506/9; 506/18

(57) ABSTRACT

Provided herein is a system and method for identifying a biomarker and producing a reagent for detecting the biomarker. The system and method comprises administering to an animal a biological sample and comparing the immune response of the animal to the immune response of another animal.

### CROSS-REFERENCE TO RELATED APPLICATIONS

This application claims the benefit of U.S. Provisional Application No. 61/436,121, filed Jan. 25, 2011, which is hereby incorporated by reference.

### BACKGROUND

Biomarkers, such as proteins, peptides, lipids, and nucleic acids, individually or in combination, are used to detect, analyze and measure a variety of biological processes. Detection of a biomarker, such as the presence, absence, or change in expression of a biomarker, in a sample of a subject can be used to relate information on a disease or condition of a subject. Biomarkers can be used to provide information on a diagnosis or prognosis of a condition or disease, disease or condition status or progression, or response to a therapeutic decision.

Thus, identification of biomarkers is an important need in providing disease or condition detection, prognostic prediction, disease or condition monitoring, disease or condition staging, therapeutic decision-making, and physiological state identification. Traditional means of identifying novel biomarkers, such as protein biomarkers, for a disease or condition are inefficient and limited by the current state of the art, which can cause a lengthy delay in between identifying the biomarker and producing reagents that can detect the new biomarker. Thus, there exists a critical need for not only the identification of novel biomarkers, but also agents for detecting the novel biomarkers with sensitivity and specificity. Antibodies are reagents that can have the sensitivity and specificity for detecting novel biomarkers with accuracy.

The disclosure can provide high-throughput, rapid identification of novel biomarkers and generation of reagents to detect these novel biomarkers, such as identification of novel protein biomarkers and rapid generation of antibodies to these novel biomarkers. These reagents can in turn be used to screen samples for the novel biomarkers in other sample. For example, antibodies to the novel biomarkers can be used to form an array that can be used to screen samples for the newly identified biomarkers.

Thus, the present disclosure meets these needs, and provides related advantages, by providing a system and method for identifying a novel biomarker and producing reagents for the detection of the newly identified biomarker.

### BRIEF SUMMARY

Provided herein are systems and methods for identifying a biomarker and producing a reagent for detecting the biomarker. The systems and methods comprise administering to an animal a biological sample and comparing the immune response of the animal to the immune response of another animal. The methods and systems can be used for high-throughput, rapid identification of novel biomarkers and generation of reagents to detect these novel biomarkers. One or more novel protein biomarkers can be identified, such as through the use of a proteome wide array. Antibodies to these novel biomarkers can be rapidly generated, for example, by using an animal that had been previously administered a composition comprising the novel biomarker.

A method of identifying one or more biomarkers comprising administering to a first animal a first biological sample and comparing an immune response from the first animal to an immune response from a second animal and identifying one or more biomarkers from a difference in the immune response from the first animal to the immune response from the second animal is provided. The second animal may be administered a second biological sample. The method can further comprise administering to the second animal the second biological sample.

Also provided herein, is a method for producing an antibody comprising administering to the first animal a first biological sample and comparing an immune response from the first animal to an immune response from a second animal and identifying one or more biomarkers from a difference in the immune response from the first animal to the immune response from the second animal. The second animal may be administered a second biological sample. The method can further comprise administering to the second animal the second biological sample. The first animal administered the first biological sample can be further administered the biomarker, and an antibody-generating cell from the animal can then be isolated for producing an antibody to the biomarker. The administration of the biological sample or biomarker can be an immunization.

A method for producing a antibodies or a library of antibodies with specificity to different biomarkers is also provided, comprising administering to the first animal a first biological sample and comparing an immune response from the first animal to an immune response from a second animal and identifying a plurality of biomarkers from a difference in the immune response from the first animal to the immune response from the second animal. The second animal may be administered a second biological sample. The method can further comprise administering to the second animal the second biological sample. The first animal administered the first biological sample can be further administered one or more of, or the plurality of biomarkers, and antibody-generating cells from the animal can then be isolated for producing a plurality of antibodies with specificity to the plurality of biomarkers. The method can further comprise generating a specificity profile for the antibody or plurality of antibodies.

The invention provides methods of profiling a protein composition of a biological sample by immunizing a first non-human animal with a first biological sample; screening an immune response from the first non-human animal using an array of proteome; comparing the immune response from the first non-human animal to an immune response from a second non-human animal immunized with, or administered a second biological sample; and identifying one or more biomarkers from a difference in the immune response from the first non-human animal to the immune response from the second non-human animal.

In any of the methods described herein, the isolated antibody-generating cell or cells can be a B-cell, or one or more B-cells. The isolated antibody-generating cell or cells can be used to generate one or more hybridomas or at least one hybridoma, such as fusing a B-cell from the first animal and with a myeloma cell. An antibody can be isolated from the hybridoma or at least one of the hybridomas. The antibody can be a monoclonal or polyclonal antibody.

In any of the methods described herein, the animal can be human or non-human. For example, the animal can be a mammal, such as a mouse, rat, rabbit, cat, dog, monkey, or goat. One or more of the biological sample(s) can be a tissue sample or bodily fluid, such as a human bodily fluid. For example, the bodily fluid can be blood, sera, plasma, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid, pre-ejaculatory fluid, female ejaculate, sweat, tears, cyst fluid, pleural fluid, peritoneal fluid, pericardial fluid, lymph, chyme, chyle, bile, interstitial fluid, menses, pus, sebum, vaginal secretion, mucosal secretion, stool water, pancreatic juice, lavage fluid from sinus cavities, bronchopulmonary aspirate, blastocyl cavity fluid, or umbilical cord blood. One or more of the biological sample(s) can comprise a cell, such as a stem cell, undifferentiated cell, differentiated cell, or cell from a diseased subject or subject with a specific condition. In any of the methods described herein, the first or second biological sample can be substantially depleted of a common serum protein, such as, but not limited to, albumin or IgG. Depletion can comprise filtration, fractionation, or affinity purification. The biological sample can be from a virus, bacterium, mycoplasma, parasite, fungus, or plant, or animal, such as a mammal, for example, a mouse, rat, rabbit, cat, dog, monkey, or goat.

A first or second biological sample can comprise disease or condition specific proteins. A first biological sample can be from a subject with a disease or condition and the second biological sample can be from a subject without a disease or condition. The disease or condition can be a cancer, inflammatory disease, immune disease, autoimmune disease, cardiovascular disease, neurological disease, infectious disease, metabolic disease, or perinatal condition. For example, the cancer can be breast cancer, ovarian cancer, lung cancer, colon cancer, colorectal cancer, prostate cancer, melanoma, pancreatic cancer, brain cancer hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer.

The first biological sample can be from a subject at one time point and the second biological sample can be from a subject at a later or earlier time point, wherein the subject can be the same or a different subject. For example, the subject may have a disease or condition, and samples can be taken as the disease or condition progresses. The first biological sample can be from a subject pretreatment and the second biological sample can be from a subject at post treatment, wherein the subject can be the same or different subject. The first biological sample can be from a subject non-responsive to treatment and the second biological sample can be from a subject responsive to a treatment. The first biological sample and second biological sample can be from the same or different species. The second biological sample can be from the same subject or from a different subject from which the first biological sample was obtained.

The comparing of immune responses can comprise comparing serum samples or supernatants from lymphoid cells or spleen cells from the first and second animals, wherein the immune responses can comprise a humoral immune response. The comparing can comprise detecting the level of the humoral responses to an antigen, wherein the antigen can be a peptide or protein. The antigen can be attached to an array. The difference in immune responses can be an increased humoral response in the first animal to an antigen as compared to a humoral response to the antigen in the second animal. Alternatively, the difference can be a decreased humoral response in the first animal to an antigen as compared to a humoral response to the antigen in the second animal. Furthermore, the detecting of the humoral response can comprise detecting antibody binding, such as an antibody from the humoral response, to the antigen or with a proteome array.

A library of antibodies can comprise a plurality of antibodies, wherein each antibody of the plurality of antibodies can specifically bind a plurality of transcription factors. In some embodiments, at least 1% to 100% of the plurality of antibodies can be antibodies produced or validated by the any of the methods described herein. In some embodiments, at least 1% to 100% of the plurality of antibodies are antibodies produced by a method other than the methods described herein. In some embodiments each antibody of the plurality of antibodies can be monospecific. In some embodiments at least 1% to 100% of the plurality of antibodies can be monospecific. In some embodiments, each antibody of the plurality of antibodies has a binding affinity of at least 10⁻⁷ M (K_{D}) for a transcription factor. In some embodiments, at least 1% to 100% of the plurality of antibodies has a binding affinity of at least 10⁻⁷ M (K_{D}) for a transcription factor. The plurality of antibodies can comprise at least 50 different antibodies.

In some embodiments, the plurality of antibodies binds at least 0.5% to 100% of transcription factors in a human proteome. In some embodiments, the transcription factors are mammalian transcription factors. In some embodiments the transcription factors are human transcription factors. In some embodiments each antibody in any library of antibodies described herein is immobilized on a substrate.

A method of validating one or more antibodies, or at least 1% to 100% of the antibodies, in any of the libraries or pluralities of antibodies described herein can comprise analyzing the one or more antibodies by a method selected from the group comprising immunoprecipitation (IP), immunohistochemistry (IHC), Western Blot (WB), Enzyme Linked Immunosorbant Assay (ELISA), immunofluorescence (IF), immunocytochemistry (ICC), Chromatin Immunoprecipitation (ChIP), siRNA knockdown, or any combination thereof. In some embodiments, the transcription factors for which an antibody has been validated by Chromatin Immunoprecipitation (ChIP) further comprises a bound consensus DNA molecule. In some embodiments, antibodies to transcription factors validated by Chromatin Immunoprecipitation (ChIP) comprise antibodies that when bound to the transcription factor do not obstruct the binding of the transcription factor to one or more consensus DNA molecules. In some embodiments the transcription factors for which an antibody as been validated is further analyzed by ChIP-sequencing (ChIP-Seq).

In some embodiments, at least 1% to 100% of the antibodies in any of the libraries of antibodies described herein, comprises antibodies validated by any of the methods described herein.

A method of identifying one or more biomarkers can comprise administering a first biological sample to a first non-human animal and a second non-human animal, wherein the first biological sample comprises a first plurality of antigens; administering a second biological sample to the first non-human animal after administering the first biological sample, wherein the second biological sample comprises a second plurality of antigens; administering a third biological sample to the second non-human animal after administering the first biological sample, wherein the third biological sample comprises a third plurality of antigens, wherein the third plurality of antigens comprises one or more additional antigens not present in the second plurality of antigens; and comparing an immune response from the first non-human animal to an immune response from the second non-human animal, thereby identifying one or more biomarkers. In some embodiments, the first plurality of antigens and the second plurality of antigens are the same, derived from the same source, or substantially overlap. In some embodiments, the first biological sample is administered prior to maturation of the immune system, during maturation of the immune system, or after maturation of the immune system in the first non-human animal, the second non-human animal or both.

A method of producing an antibody can comprise administering a first biological sample to a first non-human animal and a second non-human animal, wherein the first biological sample comprises a first plurality of antigens; administering a second biological sample to the first non-human animal after administering the first biological sample, wherein the second biological sample comprises a second plurality of antigens; administering a third biological sample to the second non-human animal after administering the first biological sample, wherein the third biological sample comprises a third plurality of antigens, wherein the third plurality of antigens comprises one or more additional antigens not present in the second plurality of antigens, comparing an immune response from the first non-human animal to an immune response from the second non-human animal, thereby identifying a biomarker; administering to the second non-human animal the biomarker; and isolating an antibody-generating cell from the second non-human animal for producing an antibody. In some embodiments, the first plurality of antigens and the second plurality of antigens are the same, derived from the same source, or substantially overlap. In some embodiments, the first biological sample is administered prior to maturation of the immune system, during maturation of the immune system, or after maturation of the immune system in the first non-human animal, the second non-human animal or both.

A method of producing antibodies with specificity to different biomarkers can comprise administering a first biological sample to a first non-human animal and a second non-human animal, wherein the first biological sample comprises a first plurality of antigens; administering a second biological sample to the first non-human animal after administering the first biological sample, wherein the second biological sample comprises a second plurality of antigens; administering a third biological sample to the second non-human animal after administering the first biological sample, wherein the third biological sample comprises a third plurality of antigens, wherein the third plurality of antigens comprises one or more additional antigens not present in the second plurality of antigens; comparing an immune response from the first non-human animal to an immune response from the second non-human animal; identifying a plurality of biomarkers from a difference in the immune response from the first non-human animal to the immune response from the second human animal; administering the second non-human animal the plurality of biomarkers; and isolating antibody-generating cells from the second non-human animal for producing antibodies with specificity to different biomarkers. In some embodiments, the first plurality of antigens and the second plurality of antigens are the same, derived from the same source, or substantially overlap. In some embodiments, the first biological sample is administered prior to maturation of the immune system, during maturation of the immune system, or after maturation of the immune system in the first non-human animal, the second non-human animal or both.

A method of profiling a protein composition of a biological sample can comprise immunizing a first biological sample to a first non-human animal and a second non-human animal, wherein the first biological sample comprises a first plurality of antigens; administering a second biological sample to the first non-human animal after administering the first biological sample, wherein the second biological sample comprises a second plurality of antigens; administering a third biological sample to the second non-human animal after administering the first biological sample, wherein the third biological sample comprises a third plurality of antigens, wherein the third plurality of antigens comprises one or more additional antigens not present in the second plurality of antigens; screening an immune response from the second non-human animal using an array of proteome; comparing an immune response from the first non-human animal to an immune response from the second non-human animal; and identifying one or more biomarkers from a difference in the immune response from the first non-human animal to the immune response from the second human animal. In some embodiments, the first plurality of antigens and the second plurality of antigens are the same, derived from the same source, or substantially overlap. In some embodiments, the first biological sample is administered prior to maturation of the immune system, during maturation of the immune system, or after maturation of the immune system in the first non-human animal, the second non-human animal or both.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a schematic of a method for identifying a biomarker and generating antibodies against the identified biomarker.

FIG. 2 depicts a silver stained polyacrylamide gel of human proteins purified as GST fusions from yeast tested for purity and quality. In order from left to right: (top) Marker, CRIP2, RUVBL2, TSN, MGMT, CRIP1, UBEC2, RNF141, SSBP4, CGorfl15, SF3B4, ZC3H7A, UMD1, TRM60, SUFU, NAP1L1, ZNF785, ANKS1, N6AMT2, CTBP1, PUF80, CBFC1, SRXN1, CGorf108, UTP6, and Marker. In order from left to right: (bottom) Marker, AK1, GOT1, ACOX1, MPP1, RAB6C, MAPK1, DOK1, SCP2, LBTD2, HSPB1, ANXA5, TMSL3, HAGH, RAB5B, FABP3, HK1, TPI1, KAD1, STIP1, NMEK1, HSPE1, UBE2K, and Marker.

FIG. 3A depicts a flow chart of recombination cloning into a destination vector.

FIG. 3B depicts a restriction enzyme digestion of 8064 constructs prepared as shown in FIG. 3A.

FIG. 4A depicts a silver stained polyacrylamide gel of human proteins purified as GST-His fusion proteins tested for purity and quality.

FIG. 4B depicts purified human proteins and control proteins spotted in duplicate on a glass slide and visualized with anti-GST antibodies

FIG. 4C depicts a region of the glass slide of FIG. 4B.

FIGS. 5A and B depicts SMMC-7721 cells stained by mAbs 1G9, and 1E3, respectively.

FIGS. 5D and E depicts CCC-HEL-1 cells stained by mAbs B4B5C5, and 2B2, respectively.

FIG. 5C depicts SMMC-7721 cells stained by ascites from un-immunized mice as negative controls.

FIGS. 5F and G depict CCC-HEL-1 cells stained by ascites from un-immunized mice as negative controls.

FIG. 6A depicts a full image of a human liver protein microarray that was probed with mAb 3A3b. Inset on the right corner illustrates detection of Pirin by 3A3b on the microarray.

FIG. 6B-E show that four more mAbs specifically recognize the following human proteins: FGL1, ORMDL2, eIF1AY, and HAb18G/CD147, respectively

FIG. 7 depicts mAb validation using immunoblot analysis.

FIG. 8 depicts IHC staining on a tissue microarray using anti-FGL1, anti-ORMDL2, anti-HAb18G, and anti-eIF1A mAbs. A-D, Normal liver, liver carcinoma, normal rectum, and recta carcinoma tissues stained by anti-FGL1 mAb, respectively. E-H, Normal liver, liver carcinoma, normal stomach, and stomach carcinoma tissues stained by anti-ORMDL2 mAb, respectively. I-L, Normal liver, liver carcinoma, normal lung, and lung carcinoma tissues stained by anti-HAb18G mAb, respectively. M-P, Normal liver, liver carcinoma, normal esophagus, and esophagus carcinoma tissues stained by anti-eIF1A mAb, respectively.

FIG. 9 depicts an overview of an antibody validation and production pipeline.

FIG. 10 depicts a pooling strategy for profiling antibody specificity.

FIG. 11 depicts a schematic of a method for identifying a biomarker and generating antibodies against the identified biomarker.

FIG. 12 depicts an anti-V5 Western Blot where mMAbs were used to immunoprecipitate the corresponding V5-tagged target proteins overexpressed in HeLa cells. Loading controls (Ponceau S and anti-beta actin immunoblotting) are shown.

FIG. 13 depicts anti-BCAP31 (left), anti-HNRNPC (center), and anti-BSG (right) Western Blot analyses. The mAbs recognize these respective native proteins. “H” indicates HeLa cell extract. “S” indicates SH-SY5Y neuroblastoma cell extract.

FIG. 14 depicts real-time detection of antigen-antibody interactions using OIRD methods on a protein microarray. A differential image of protein microarray containing mouse and rat IgGs after probing with anti-mouse IgG is shown in A. B illustrates that on-rates can be measured with binding curves obtained by realtime monitoring.

FIG. 15 depicts an experiment to test for an anti-transcription factor antibody's ability to perform chromatin immunoprecipitations (ChIP). Shown is a silver stained polyacrylamide gel where anti-HNRPC was used to immunoprecipitate a chromatin preparation.

FIG. 16 depicts an IgG-secreting colony growing in methylcellulose that contains anti-IgG 488.

FIG. 17 depicts an immunofluorescence image where double-labeling of a clone secreting antibody against GST was performed. Colonies were grown in MC containing GST labeled with DyLight-549, plus anti-mouse IgG labeled with DyLight-488, and examined with excitation wavelengths of 549 nm (left), 488 nm (center), or white light (right).

FIG. 18 depicts an immunocytochemistry (ICC) assessment of the ability of antibodies to bind proteins in fixed cells. ICC images of mAb staining are shown. The assessment was performed using the following antibodies with the indicated cells from left to right: anti-BIRC7, HeLa; anti-BCAP31, MCF7; anti-BRCC36, HepG2; HNRPC, mixed tumor cells

FIG. 19 depicts a liquid chromatography-mass spectrometry (LC-MS/MS) validation of protein identification on purified recombinant targets. Panel A shows the total ion chromatogram of Folate 1 receptor (FOLR1) during a liquid chromatography (LC) run. Panel B shows the peptides identified by MS/MS.

FIG. 20 depicts the Tet Expression Vector system. Panel A depicts a human ORF regulated expression vector under construction. FV is FLAG-V5 tag; GST is GST flanked by in-frame loxP sites (arrowheads); boxed X's are Gateway sites shown in the post-recombination state after an ORF has been subcloned; pA is SV40 polyadenylation signal; oriP is high copy viral origin; Puro is puromycin resistance gene. Black boxes are FLP sites allowing the entire FV-GST tag to be removed by site-specific recombination with FRT in vitro or in vivo, allowing expression of the protein from its native N terminus (AUG2). The base vector is pCEP4. NotI and SgrAI sites are unique. Horizontal dashed line indicates new segment to be made. Panel B is a Western Blot depicting expression of a mammalian ORF in the current base vector (Tetp-CEP4-Puro in Tet-On HeLa cells (left) and Tet-Off HeLa cells (center). (right; −, + refers to Doxycycline addition). ARRPPo ia control endogenous protein. Panel C depicts Tet-ON cells with varying Dox concentration (ng/mL).

FIG. 21 depicts a demonstration of a single plasmid knockdown validation strategy. Panel A shows a high-throughput cloning strategy based can work in large plasmids. BstZ17I was used as subcloning site. Panel B shows this approach can be used to subclone shRNA into pCEP-Puro expressing any ORF. Either an L1 reporter construct (insert size 6 kb) or eGFP was used. Panel C shows immunofluorescence images demonstrating the single plasmid systems can efficiently knock down expression of the CMV promoter-expressed genes. eGFP fluorescence is shown. Panels D and E show shRNA expressed can silence an endogenous gene (PABPC1) detected by RT-PCR (D) or immunoblotting (E).

FIG. 22 depicts a schematic for chromatin immunoprecipitaion and sequencing (ChIP-seq) work flow.

FIG. 23 depicts characteristic peak shapes for a transcription factor binding sites using CisGenome. Reads obtained from both ends of an immunoprecipitated DNA fragment are shown. 5′ reads aligned in the forward orientation and 3′ reads aligned in the reverse orientation are depicted. From top to bottom, the first track shows the number of 5′ reads aligning to a region, the number of 3′ reads aligning to a region, the 5′ read counts in a sliding window of 100 bp, the 3′ read counts in a sliding window of 100 bp.

FIG. 24 depicts an SDS-PAGE analysis of full-length human proteins expressed in E. coli.

FIG. 25 depicts a cell microarrays (CMA) consisting of about 40 pancreatic cancer cells

FIG. 26 depicts immunohistochemistry (IHC) staining of CMAs using an antibody against CD44.

FIG. 27 depicts immunohistochemistry (IHC) staining of CMAs using an antibody against E-Cadherin.

FIG. 28 is a table of some of the monoclonal antibodies of high quality developed using the methods described herein (Table 1) including antibodies against sequence-specific DNA binding proteins.

### DETAILED DESCRIPTION

The present disclosure provides systems and methods for identifying a biomarker. The systems and methods disclosed herein can also be used to identify one biomarker or a plurality of biomarkers (e.g. profiling a composition of a complex sample). The biomarker can be a novel or new biomarker. The biomarker can be a biomarker that has not been previously identified or detected in a sample. The biomarker may be previously identified for a different disease or condition, but not previously identified as a biomarker for another disease or condition and is therefore novel for the disease or condition. The biomarker can be a novel biomarker for a condition or disease, such as for the detection, diagnosis, theranosis, or prognosis of a disease or condition. The biomarker can be a novel biomarker for monitoring a condition or disease, monitoring a therapeutic response, or for the selection of a therapeutic. The biomarker can be any component produced by a biological organism. For example, the biomarker can be a protein, peptide, lipid, or nucleic acid, such as RNA or DNA. The present invention provides systems and methods for generating a profile of the protein composition of a sample.

Also provided herein is a method and system for producing reagents for the detection of the biomarker. The reagent can be a therapeutic. For example, the reagent to a biomarker can be an antibody to the biomarker. The antibody can be used to detect the biomarker, or can be a therapeutic. The reagents can be also be used to produce an array, such as an array for detecting a plurality of biomarkers. For example, the reagents can comprise antibodies, which can be used to produce, generate, or form an antibody library. In one embodiment, the reagents can comprise transcription factor specific antibodies, which can be used to produce, generate, or form an antibody library to one or more transcription factors. The antibodies can be attached or linked to an array.

A method of identifying one or more biomarkers can comprise administering to a first animal a first biological sample and comparing an immune response from the first animal to an immune response from a second animal. The method can further comprise identifying one or more biomarkers from a difference in the immune response from the first animal to the immune response from the second animal. The second animal may be administered a second biological sample. The method can further comprise administering to the second animal the second biological sample. Also provided herein, is a method for producing an antibody comprising administering to the first animal the biomarker. The first animal administered the first biological sample can be further administered the biomarker, such as a protein biomarker. An antibody-generating cell from the animal can then be isolated for producing an antibody to the biomarker. The administration can be an immunization.

Methods of generating a profile can comprise immunizing an animal with a first biological sample and profiling the immune response against a protein array. The immune response can be the production of antibodies. By using a protein array to measure antibody production in an animal in response to immunization with a biological sample the methods provide herein decode a fluid antibody array generated by the animal (e.g. a mouse). This fluid antibody array can then be used to generate reagents as described herein, for instance, to generate an antibody array. In some embodiments, an antibody array comprises one or more transcription factor specific antibodies. In one embodiment, an antibody array comprises only one or more antibodies specific to transcription factors.

The animal can be a human or non-human animal, such as a mammal. The mammal can be a bovine, avian, canine, equine, feline, ovine, porcine, or primate animal. For example, the mammal can be a mouse, rat, rabbit, cat, dog monkey, or goat. The animals can be clones of each other, that is, the animals are identical. The animals can be identical in case variability amongst individuals is a concern.

The first or second biological sample can be an in vitro sample, such as one or more purified proteins, or an in vivo sample. The one or more purified proteins can be produced by any of the methods described herein or by methods known to one skilled in the art. For example, the first or second biological sample can be a recombinant protein produced in a host, or from a cell line. In some embodiments, the biological sample can be one or more biomarkers. In some embodiments, the biological sample can be one or more non-biomarkers (i.e., for tolerization). Alternatively, the first or second biological sample can be from a subject, such as a human or non-human subject. The subject can be a mammal, such as a bovine, animal, canine, equine, feline, ovine, porcine, or primate animal. For example, the mammal can be a mouse, rat, rabbit, cat, dog monkey, or goat. In another embodiment, the biological sample can be from a virus, bacterium, mycoplasma, parasite, fungus, or plant subject.

The biological sample can be directly obtained from the subject or derived from the subject, such as from a culture of the subject's sample. For example, the biological sample can be a tissue sample or bodily fluid, such as a human bodily fluid. The bodily fluid can be blood (such as peripheral blood), serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid, pre-ejaculatory fluid, female ejaculate, sweat, fecal matter, hair, tears, cyst fluid, pleural fluid, peritoneal fluid, pericardial fluid, lymph, chyme, chyle, bile, interstitial fluid, menses, pus, sebum, vaginal secretion, mucosal secretion, stool water, pancreatic juice, lavage fluid from sinus cavities, bronchopulmonary aspirate, blastocyl cavity fluid, or umbilical cord blood. The biological sample can also be blastocyl cavity or umbilical cord blood. The biological sample can be a tissue sample or biopsy. The first and second biological samples can be of the same type, such as both being sera, or alternatively, they can be of different types, such as one being sera the other being CSF.

The biological sample can comprise a cell. The biological sample can be from any tissue or be of any cell type. For example, the biological sample can comprise a breast, ovarian, lung, colon, prostate, skin, pancreatic, neural, blood, hepatic, endometrial, esophageal, gastrointestinal, renal, or gastric tissue or cell. The cell can be a stem cell, differentiated cell, or undifferentiated cell.

The biological sample can comprise a plurality of antigens that can comprise purified or non-purified samples, such as purified or non-purified tissues, fluids, cells, proteins, peptides, or nucleic acids. The plurality of antigens can comprise a biological sample. In one embodiment, the biological sample can comprise one or more transcription factors. Transcription factors can be cell-specific markers, and can show nuclear localization, which can allow for quantification of cell subtypes. Also contemplated is an array of antibodies specific to one or more proteins or one or more proteins of a biological sample. An array of antibodies can comprise antibodies specific to at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the proteins sharing one or more common attributes in the proteome of a species, for example, transcription factors. The common attribute can be, for example, a common structural feature, a common location, a common biological process, or a common molecular function, such as transcription factors. In some embodiments, a library or array can comprise a plurality of antibodies specific to a plurality of antigens in which the library or array represents at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of all of the antigens that share a common attribute, such as transcription factors. In some embodiments, a library or array can comprise a plurality of antibodies specific to a plurality of transcription factors in which the library or array represents at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of all of the transcription factors of the proteome of a species.

In some embodiments, the antibodies, such as mMAbs, identified using the methods described herein can be used to create multiplex assays or arrays of affinity molecules, such as antibodies to transcription factors. The arrays or multiplex assays can comprise a plurality of antibodies specific to at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the proteins sharing one or more common attributes. In some embodiments, arrays or multiplex assays can comprise a plurality of antibodies specific to a plurality of transcription factors in which the array or multiplex assay represents at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of all of the transcription factors of the proteome of a species.

The antibodies of the arrays can be identified or determined using the methods described herein. The antibodies of the arrays can be identified or determined using any other suitable method known in the art. In some embodiments, the arrays or multiplex assays can comprise monospecific antibodies to at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the proteins sharing one or more common attributes, such as transcription factors, as identified by the methods described herein. In some embodiments, the arrays or multiplex assays can comprise monospecific antibodies to at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the proteins sharing one or more common attributes, such as transcription factors, as identified by any other suitable method known in the art.

In some embodiments, at least 1% to 100% of the antibodies of the libraries, arrays, or multiplex assays can be validated by one or more of the methods described herein, for example, at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the antibodies of the libraries, arrays, or multiplex assays can be validated by one or more of the methods described herein. Such methods, include, but are not limited to immunoprecipitation (IP), immunohistochemistry (IHC), Western Blot (WB), Enzyme Linked Immunosorbant Assay (ELISA), immunofluorescence (IF), immunocytochemistry (ICC), Chromatin Immunoprecipitation (ChIP), siRNA knockdown, or any combination thereof. In some embodiments, at least 1% to 100% of the antibodies of the libraries, arrays, or multiplex assays can be validated by other methods known in the art, for example, at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the antibodies of the libraries, arrays, or multiplex assays can be validated by other methods known in the art. Transcription factors for which an antibody has been validated by Chromatin Immunoprecipitation (ChIP) can be bound to consensus DNA molecule. Antibodies to transcription factors validated by Chromatin Immunoprecipitation (ChIP) can comprise antibodies that when bound to the transcription factor do not obstruct the binding of the transcription factor to one or more consensus DNA molecules. The transcription factors for which an antibody as been validated, can be further analyzed by ChIP-sequencing (ChIP-Seq).

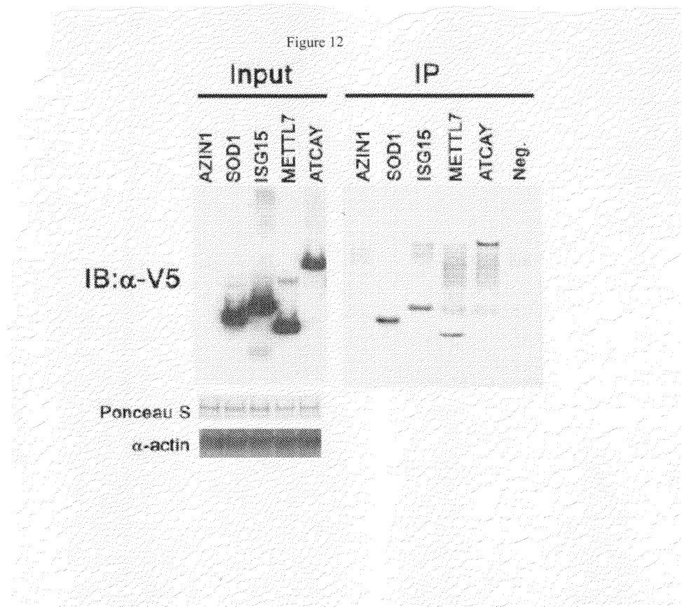

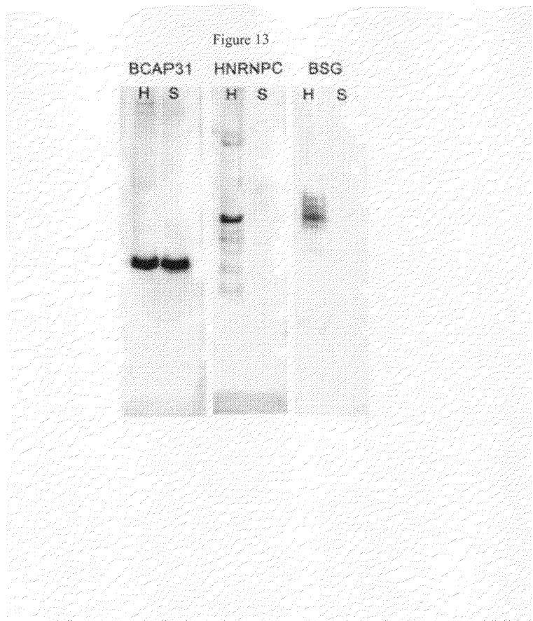

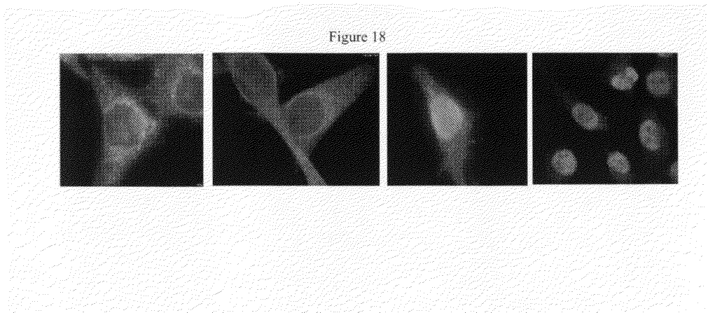

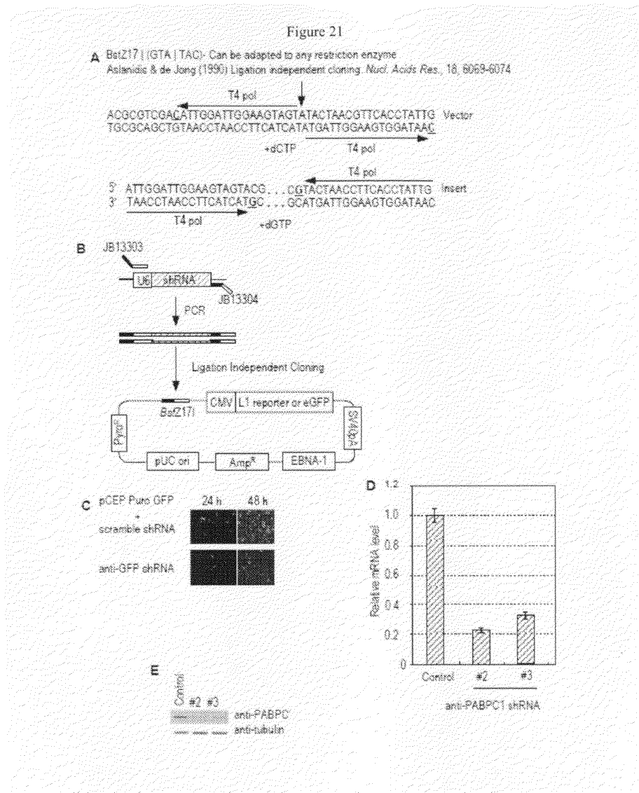

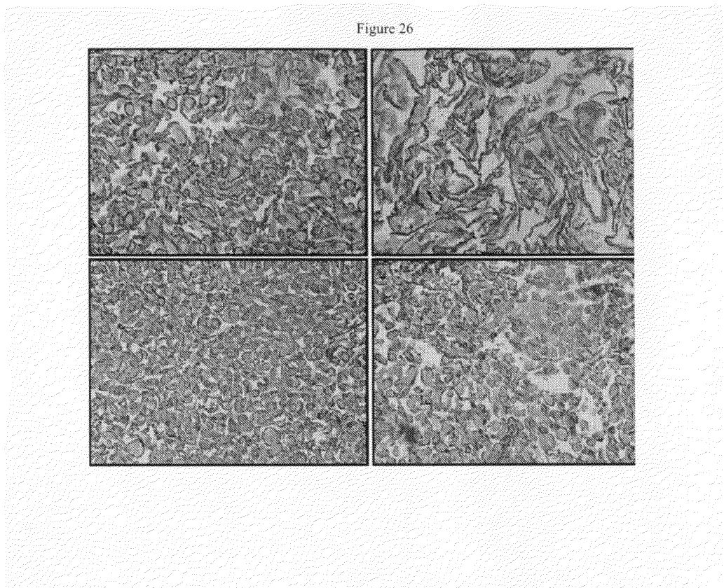

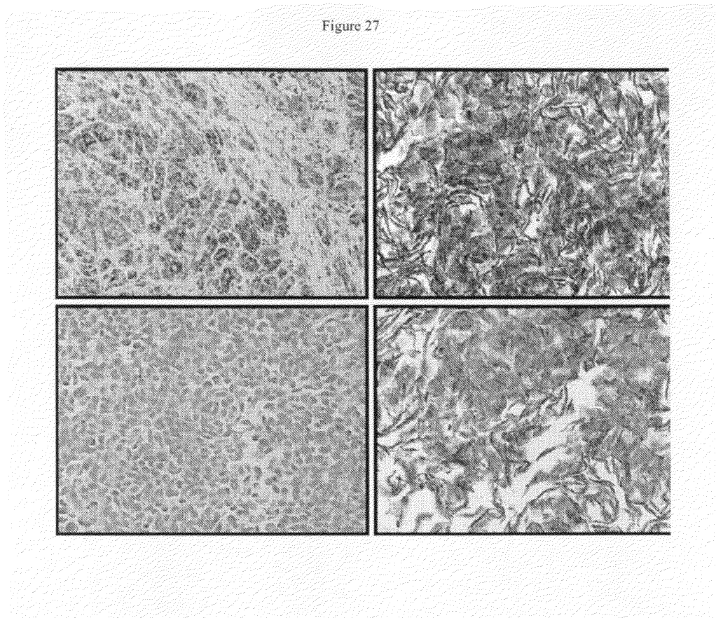

In some embodiments, the arrays or multiplex assays can comprise monospecific antibodies to at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of all of the transcription factors of the proteome of a species. In some embodiments, the monospecific antibodies to at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of all of the transcription factors of the proteome of a species can be validated by Chromatin Immunoprecipitation (ChIP) (for example, high-density microarray hybridization (ChIP-Chip) and deep sequencing (ChIP-Seq)), immunoprecipitations (IP) (FIG. 12 and FIG. 13), immunohistochemistry (IHC) (FIG. 26 and FIG. 27), Western Blot (WB) (FIG. 12 and FIG. 13), Enzyme Linked Immunosorbant Assay (ELISA), immunofluorescence (IF), immunocytochemistry (ICC) (FIG. 18), utilizing blocking peptides or molecules, siRNA knockdown experiments (FIG. 21), or any combination thereof.

The monospecific antibodies of the libraries, arrays or multiplex assays can be validated and/or their specificities can be determined or evaluated by immunoprecipitations (IP), immunohistochemistry (IHC), Western Blot (WB), Enzyme Linked Immunosorbant Assay (ELISA), immunofluorescence (IF), immunocytochemistry (ICC) (FIG. 18), Chromatin Immunoprecipitation (ChIP) (for example, high-density microarray hybridization (ChIP-Chip) and deep sequencing (ChIP-Seq)), utilizing blocking peptides or molecules, siRNA knockdown experiments (FIG. 21), or any combination thereof. A validated antibody, such as a monospecific antibody, can be specific for a selected target, for example, if a band or bands at the known molecular weight for the target is observed by WB, such as when the sample comprises numerous targets, for example, cell lysates. The presence of multiple bands or bands not at the proper molecular weight could represent the same target at different post-translational modification status, breakdown products, or splice variants.

Controls for validation experiments can comprise negative and positive controls. Non-limiting examples of controls can comprise, samples known not to express the target, samples overexpressing the target, sample transfected with the target, target knockout samples, samples with siRNA or shRNA to the target (FIG. 21), isotyped samples, samples pretreated with an agent (i.e., phosphatase treatment for phospho-specificity) or any combination thereof.

Another aspect of the present invention is a library of antibodies comprising a plurality of antibodies, wherein each antibody of the plurality of antibodies can specifically bind a plurality of transcription factors. At least 1% to 100% of the antibodies of the library can be produced by any of the methods described herein. For example, at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the antibodies can be produced by any of the methods described herein. At least 1% to 100% of the antibodies of the library can be produced by a method other than the methods described herein. For example, at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55% 60%, 65% 70%, 75% 80%, 85% 90%, 95% or 100% of the antibodies can be produced by a method other than the methods described herein.

At least one of the antibodies in the library can be monospecific. At least 1% of the antibodies in the library can be monospecific. For example, at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the antibodies in the library can be monospecific. In one embodiment, each of the antibodies in the library or plurality of antibodies can be monospecific.

At least one of the antibodies in the library can have a binding affinity of at least 10⁻⁷ M (K_{D}), such as at least 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or 10⁻¹⁶ M, for its target. At least 1% of the antibodies in the library can be monospecific and at least one of the antibodies in the library can have a binding affinity of at least 10⁻⁷ M (K_{D}). For example, at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the antibodies in the library can be monospecific and at least one of the antibodies in the library can have a binding affinity of at least at least 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or 10⁻¹⁶ M.

A library of antibodies can comprise at least 50 antibodies. For example, a library of antibodies can comprise at least 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 antibodies.

In one embodiment, a library of antibodies can comprise at least 50 different antibodies. For example, a library of antibodies can comprise at least 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 different antibodies.

In some embodiments, a library of antibodies can comprise at least 2 of the same one or more antibodies. For example, a library of antibodies can comprise at least 2, 3, 4, 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 of the same one or more antibodies.

A library of antibodies can comprise a plurality of antibodies that bind at least about 0.5% to 100% of the transcription factors in a proteome, such as a human proteome. For example, a library of antibodies can comprise a plurality of antibodies that bind at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the transcription factors in a proteome. In one embodiment a library of antibodies can comprise a plurality of antibodies that bind at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the transcription factors in a human proteome.

The proteome can be a human or non-human proteome. The proteome can be mammalian, such as a bovine, avian, canine, equine, feline, ovine, porcine, or primate proteome. For example, the mammalian proteome can be a human, mouse, rat, rabbit, cat, dog monkey, or goat proteome. In another embodiment, the proteome can be a virus, bacterium, mycoplasma, parasite, fungus, or plant proteome.

Any of the libraries of antibodies, pluralities of antibodies, or parts thereof, described herein, can be an array of antibodies. An array can comprise a library of antibodies as described herein. On or more of the antibodies, a plurality of antibodies, or each antibody can be immobilized on a substrate. At least 1%, for example, 10%, 20%, 30%, 40%, 50%, 60%, 70%, 80%, 90%, 95%, 99%, or 100% of the antibodies in a library of antibodies can be immobilized on a substrate. The substrate can be planar or a particle, comprise a solid or porous material. The immobilization can be reversible or irreversible.

At least 1% to 100% of the antibodies of the array can be produced by the methods described herein. For example, at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the antibodies can be produced by the methods described herein. At least 1% to 100% of the antibodies of the array can be produced by a method other than the methods described herein. For example, at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, of the antibodies can be produced by a method other than the methods described herein.

At least one of the antibodies in the array can be monospecific. For example, at least 2, 3, 4, 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 of the antibodies in the array can be monospecific. At least 1% of the antibodies in the array can be monospecific. For example, at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the antibodies in the array can be monospecific. In one embodiment, each of the antibodies in the array or plurality of antibodies can be monospecific.

At least one of the antibodies in the array can have a binding affinity of at least 10⁻⁷ M (K_{D}), such as at least 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or 10⁻¹⁶ M, for its target. At least one of the antibodies in the array can have a binding affinity of at least 10⁻⁷ M (K_{D}) for a transcription factor. For example, at least one of the antibodies in the array can have a binding affinity of at least at least 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or 10⁻¹⁶ M, for a transcription factor.

An array of antibodies can comprise at least 50 antibodies. For example, an array of antibodies can comprise at least 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 antibodies.

In one embodiment, an array of antibodies can comprise at least 50 different antibodies. For example, an array of antibodies can comprise at least 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 different antibodies.

An array of antibodies can comprise a plurality of antibodies that bind at least 0.5% to 100% of the transcription factors in a proteome, such as a human proteome, For example, an array of antibodies can comprise a plurality of antibodies that bind at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the transcription factors in a proteome, such as a human proteome.

The proteome can be a human or non-human proteome. The proteome can be mammalian, such as a bovine, avian, canine, equine, feline, ovine, porcine, or primate proteome. For example, the mammalian proteome can be a human, mouse, rat, rabbit, cat, dog monkey, or goat proteome. In another embodiment, the proteome can be a virus, bacterium, mycoplasma, parasite, fungus, or plant proteome.

Any of the antibodies or any subset of the antibodies in a library, array, or multiplex assay can bind a native form or denatured form of its transcription factor; be a monoclonal or polyclonal antibody; be an immunoprecipitating antibody; be an IgG, IgA, IgD, IgE, or IgM antibody or antibody of IgG, IgA, IgD, IgE, or IgM isotype; or any combination thereof.

Binding can be detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, chromatin immunoprecipitations assays (ChIP) and immunoelectrophoresis assays. In some embodiments, binding can be detected by one or more of the techniques disclosed herein.

Antibody binding can be detected by detecting a label on the primary antibody. Alternatively, the primary antibody can be detected by detecting binding of a secondary antibody or reagent to the primary antibody. For example, the secondary antibody can be labeled. In some embodiments, an automated detection assay or high-throughput system is utilized. For example, in a capture micro-enzyme-linked immunosorbent assay (ELISA), an antibody/antigen reaction is made measurable by immobilization of the antibody and subsequent direct or indirect colorimetric, fluorescent, luminescent or radioactive detection of bound, labeled antigens. For example, the antigen can be labeled by biotin or other labels, which will allow downstream detection.

The immobilized antibodies can bind to a single antigenic determinant present. The antigenic determinant can be labeled, such as through labeling of the biomarker comprising the antigenic determinant The specificity of this reaction will permit quantification in the ELISA measurements. The ELISA reaction can be used in a high throughput format to screen hybridoma supernatants. Screening assays built on other principles than an ELISA can be deployed (e.g., antibody microarrays, high-throughput screening based on MALDI/MS and/or multi-channel capillary electrophoresis). ELISA or microarray data can be evaluated, e.g., by published methods. The goal of the data analysis process can be the selection of hybridoma supernatants that show the best collection with an important clinical parameter and can be specific to one of the analyte groups.

In some embodiments an array (or microarray) or multiplex assay can comprise a library of antibodies or a plurality of antibodies and a substrate. In some embodiments, each antibody is immobilized on a substrate. The antibody may be reversibly or irreversibly immobilized on the substrate. The substrate can be planar or a particle and can comprise a solid or porous material. The substrate may be either organic or inorganic, biological or non-biological, or any combination of these materials.

The substrate can transparent or translucent. The portion of the surface of the substrate on which the patches reside can be flat and firm or semi-firm. Numerous materials are suitable for use as a substrate. The substrate can comprise silicon, silica, glass, or a polymer. For instance, the substrate can comprise a material selected from a group consisting of silicon, silica, quartz, glass, controlled pore glass, carbon, alumina, titanium dioxide, germanium, silicon nitride, zeolites, and gallium arsenide. Many metals such as gold, platinum, aluminum, copper, titanium, and their alloys can also be used for substrates of the array. In addition, many ceramics and polymers can also be used as substrates. Polymers which can be used as substrates include, but are not limited to, the following: polystyrene; poly(tetra)fluorethylene; (poly)vinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polyhydroxyethylmethacrylate; polydimethylsiloxane; polyacrylamide; polyimide; co-block-polymers; and Eupergit® Photoresists, polymerized Langmuir-Blodgett films, and LIGA structures

A microarray of distinct antibodies can be bound on a glass slide coated with a polycationic polymer. A substrate can be formed according to another aspect of the invention, and intended for use in detecting binding of target molecule to one or more distinct antibodies. In one embodiment, the substrate includes a glass substrate having formed on its surface, a coating of a polycationic polymer, preferably a cationic polypeptide, such as poly-lysine or poly-arginine. Formed on the polycationic coating is a microarray of distinct biopolymers, each localized at known selected array regions, such as spots or regions.

The slide may be coated by placing a uniform-thickness film of a polycationic polymer, e.g., poly-L-lysine, on the surface of a slide and drying the film to form a dried coating. The amount of polycationic polymer added can be sufficient to form at least a monolayer of polymers on the glass surface. The polymer film can be bound to surface via electrostatic binding between negative silyl-OH groups on the surface and charged amine groups in the polymers. Poly-l-lysine coated glass slides can be obtained commercially, e.g., from Sigma Chemical Co. (St. Louis, Mo.).

A suitable microarray substrate can also be made through chemical derivatization of glass. Silane compounds with appropriate leaving groups on a terminal Si will covalently bond to glass surfaces. A derivatization molecule can be designed to confer the desired chemistry to the surface of the glass substrate. An example of such a bifunctional reagent is amino-propyl-tri(ethoxy)silane, which reacts with glass surfaces at the tri(ethoxy)silane portion of the molecule while leaving the amino portion of the molecule free. Surfaces having terminal amino groups are suitable for adsorption of biopolymers in the same manner as poly-lysine coated slides. The identity of the terminal surface group can be modified by further chemical reaction. For example, reaction of the terminal amine in the above example with glutaraldehyde results in a terminal aldehyde group. Further layers of modification may be applied to achieve the desired reactivity before spotting the microarray, such as by application of a Protein A or Protein G solution to the silynated glass. Additional surfaces that bind polypeptides are nitrocellulose-coated glass slides, available commercially from Schleicher and Schuell, and protein-binding plastics such as polystyrene.

The spotted antibodies can be attached by either adsorption or covalent bonding. Adsorption occurs through electrostatic, hydrophobic, Van der Waals, or hydrogen-bonding interactions between the spotted polypeptide and the array substrate. Simple application of the polypeptide solution to the surface in an aqueous environment can be sufficient to adsorb the polypeptide. Covalent attachment can be achieved by reaction of functional groups on the polypeptide with a chemically activated surface. For example, if the surface has been activated with a highly reactive electrophilic group such as an aldehyde or succinimide group, unmodified polypeptides react at amine groups, as at lysine residues or the terminal amine, to form a covalent bond.

To form the microarray, defined volumes of distinct biopolymers can be deposited on the polymer-coated slide using any suitable method known in the art. According to an important feature of the substrate, the deposited antibodies can remain bound to the coated slide surface non-covalently when an aqueous sample is applied to the substrate under conditions that allow binding of labeled ligands in the sample to cognate binding partners in the substrate array.

In some embodiments, each microarray contains at least 50, 60, 70, 80, 90, 100, 150, 200, 250, 300, 350, 400, 450, 500, 600, 700, 800, 900, or 1000 distinct antibodies, the same antibodies, or a combination thereof per surface area of less than about 1 cm². In one embodiment, the microarray contains 50, 60, 70, 80, 90, 100, 150, 200, 250, 300, 350 or 400 regions in an area of about 16 mm², or 2.5×10³ regions/cm². The antibodies in each microarray region can be present in a defined amount between about 0.1 femtomoles and 100 nanomoles.

Also in a preferred embodiment, the biopolymers can have lengths of at least about 50 units, e.g. amino acids, nucleotides, etc., i.e., substantially longer than polymers which can be formed in high-density arrays by various in situ synthesis schemes.

The microarrays of the invention can be used in medical diagnostics, drug discovery, molecular biology, immunology and toxicology. Microarrays of immobilized antibodies prepared in accordance with the invention can be used for large scale binding assays in numerous diagnostic and screening applications. The multiplexed measurement of quantitative variation in levels of large numbers of targets (e.g. proteins) allows the recognition of patterns defined by several to many different targets (e.g., proteins). Many physiological parameters and disease-specific patterns can be simultaneously assessed. One embodiment involves the separation, identification and characterization of proteins present in a biological sample. For example, by comparison of disease and control samples, it is possible to identify “disease specific proteins”. These proteins can be used as targets for drug development or as molecular markers of disease.

Antibody arrays can be used to monitor the expression levels of proteins, such as transcription factors, in a sample where such samples can include biopsy of a tissue of interest, cultured cells, microbial cell populations, biological fluids, including blood, plasma, lymph, synovial fluid, cerebrospinal fluid, cell lysates, culture supernatants, amniotic fluid, etc., and derivatives thereof. Of particular interest are clinical samples of biological fluids, including blood and derivatives thereof, cerebrospinal fluid, urine, saliva, lymph, synovial fluids, etc. Such measurements may be quantitative, semi-quantitative, or qualitative. Where the assay is to be quantitative or semi-quantitative, it will preferably comprise a competition-type format, for example between labeled and unlabeled samples, or between samples that are differentially labeled.

Assays to detect the presence of target molecules to the immobilized polypeptides may be performed as follows, although the methods need not be limited to those set forth herein and include any suitable method known in the art.

Samples, fractions or aliquots thereof can be added to an array or microarray comprising the antibodies. Samples can comprise a wide variety of biological fluids or extracts as described above. Preferably, a series of standards, containing known concentrations of control samples can be assayed in parallel with the samples or aliquots thereof to serve as controls. The incubation time should be sufficient for target molecules to bind the samples, such as polypeptides, such as from about 0.1 to 3 hr, usually 1 hr, but could be as long as one day or longer.

After incubation, the insoluble support can be washed of non-bound components. A dilute non-ionic detergent medium at an appropriate pH, such as a pH between 7-8, can be used as a wash medium. From one to six washes can be employed, with sufficient volume to thoroughly wash non-specifically bound proteins present in the sample.

The target itself may be labeled with a detectable label, and the amount of bound label directly measured. Alternatively, the labeled sample may be mixed with a differentially labeled, or unlabeled sample in a competition assay. In yet another embodiment, the target itself is not labeled, but a second stage labeled reagent is added in order to quantitate the amount of target present.

Examples of labels that permit direct measurement of ligand binding include radiolabels, such as ³H or ¹²⁵I, fluorescers, dyes, beads, chemilumninescers, colloidal particles, and the like. Suitable fluorescent dyes are known in the art, including fluorescein isothiocyanate (FITC); rhodamine and rhodamine derivatives; Texas Red; phycoerythrin; allophycocyanin; 6-carboxyfluorescein (6-FAM); 2′,7′-dimethoxy-4′,5′-dichloro-6-carboxyfluorescein (JOE); 6-carboxy-X-rhodamine (ROX); 6-carboxy-2′,4′,7′,4,7-hexachlorofluorescein (HEX); 5-carboxyfluorescein (5-FAM); N,N,N′,N′-tetramethyl-6-carboxyrhodamine (TAMRA); sulfonated rhodamine; Cy3; Cy5; etc. Preferably the compound to be labeled is combined with an activated dye that reacts with a group present on the ligand, e.g. amine groups, thiol groups, aldehyde groups, etc.

Particularly where a second stage detection is performed, for example by the addition of labeled antibodies that recognize the target, the label can be a covalently bound enzyme capable of providing a detectable product signal after addition of suitable substrate. Examples of suitable enzymes for use in conjugates include, but are not limited to, horseradish peroxidase, alkaline phosphatase, malate dehydrogenase and the like. Where not commercially available, such antibody-enzyme conjugates can be readily produced by techniques known to those skilled in the art. The second stage binding reagent can be any compound that binds the target molecules with sufficient specificity such that it can be distinguished from other components present. In a preferred embodiment, second stage binding reagents are antibodies specific for the sample, either monoclonal or polyclonal sera, e.g. mouse anti-human antibodies, etc. For an amplification of signal, the sample may be labeled with an agent such as biotin, digoxigenin, etc., where the second stage reagent will comprise avidin, streptavidin, anti-digoxigenin antibodies, etc. as appropriate for the label.

Microarrays can be scanned to detect binding of molecules, analytes, or targets, e.g. by using a scanning laser microscope, by fluorimetry, a modified ELISA plate reader, etc. For example, a scanning laser microscope may perform a separate scan, using the appropriate excitation line, for each of the fluorophores used. The digital images generated from the scan are then combined for subsequent analysis. For any particular array element, the ratio of the fluorescent signal with one label can be compared to the fluorescent signal from the other label DNA, and the relative abundance can be determined

The microarrays and methods of detecting target molecules may be used for a number of screening, investigative and diagnostic assays. In one application, an array of antibodies can be bound to total protein from an organism to monitor protein expression for research or diagnostic purposes. Labeling total protein from a normal cell with one color fluorophore and total protein from a diseased cell with another color fluorophore and simultaneously binding the two samples to the same array allows for differential protein expression to be measured as the ratio of the two fluorophore intensities. This two-color experiment can be used to monitor expression in different tissue types, disease states, response to drugs, or response to environmental factors.

In screening assays, for example to determine whether a protein or proteins are implicated in a disease pathway or are correlated with a disease-specific phenotype, measurements can be made from cultured cells. Such cells may be experimentally manipulated by the addition of pharmacologically active agents that act on a target or pathway of interest. This application can be important for elucidation of biological function or discovery of therapeutic targets.

For many diagnostic and investigative purposes it can be useful to measure levels of target molecules, e.g. proteins, in blood or serum. This application can be important for the discovery and diagnosis of clinically useful markers that correlate with a particular diagnosis or prognosis. For example, by monitoring a range of antibody or T cell receptor specificities in parallel, one may determine the levels and kinetics of antibodies during the course of autoimmune disease, during infection, through graft rejection, etc. Alternatively, novel protein markers associated with a disease of interest can be developed through comparisons of normal and diseased blood sample, or by comparing clinical samples at different stages of disease.

Information on the protein expression in a genome of an organism can have a wide variety of applications, including but not limited to, diagnosis and treatment of diseases in a personalized manner (also known as “personalized medicine”) by association with phenotype such as onset, development of disease, disease resistance, disease susceptibility, drug response, or any combination thereof. Identification and characterization of the proteins relevant to biological pathways in a genome of an organism in terms of cell- or tissue-specificity can also aid in the design of transgenic expression constructs for therapy with enhanced therapeutic efficacy and/or reduced side effects. Identification and characterization of protein expression in terms of cell- or tissue-specificity can also aid in the development of function markers for diagnosis, prevention and treatment of diseases. “Disease” includes but is not limited to any condition, trait or characteristic of an organism that it is desirable to change. For example, the condition may be physical, physiological or psychological and may be symptomatic or asymptomatic.

In another embodiment of the invention, the antibody arrays are used to detect post-translational modifications in proteins, which is important in studying signaling pathways and cellular regulation. Post-translational modifications can be detected using antibodies specific for a particular state of a protein, such as phosphorylated, glycosylated, farnesylated, etc.

The detection of these interactions between ligands and polypeptides can lead to a medical diagnosis. For example, the identity of a pathogenic microorganism can be established unambiguously by binding a sample of the unknown pathogen to an array containing many types of antibodies specific for known pathogenic antigens.

### Kits

In one embodiment, a kit comprising a library of antibodies is provided. In some embodiments, the library of antibodies can be arrayed in a support, e.g., 96 or 384 wells. In one embodiment, a kit comprises a microarray of antibodies, such as a microarray of antibodies to transcription factors. The kit may further include: reporter assay substrates; reagents for induction or repression of a particular biological pathway (cytokines or other purified proteins, small molecules, cDNAs, siRNAs, etc.), and/or data analysis software.

In addition, kits are provided which comprise reagents and instructions for performing methods of the present invention, or for performing tests or assays utilizing any of the compositions, libraries, arrays, or assemblies of articles of the current disclosure. The kits can further comprise buffers, enzymes, adaptors, labels, secondary antibodies and instructions necessary for use of the kits, optionally including troubleshooting information.

In yet another embodiment, the kit may comprise a library of antibodies, such as described herein, and a library of antigens, such as a proteome (or part thereof) of an organism. The kit may comprise at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the transcription factors in a proteome, such as a human proteome. The library of antigens can represent a substantial portion or all of the transcription factors of a proteome, such as a bacterial, viral, fungal, mammalian or human proteome. The library of antigens can represent a substantial portion or all of the transcription factors of a proteome of an insect or mammal, such as a mouse, rat, rabbit, cat, dog, monkey, goat, or human.

The biological sample can be treated prior to being used to immunize an animal. For example, the biological sample can be enriched or purified, such as by substantially depleting an abundant protein or contaminant, or a number of different proteins or contaminants, from the biological sample, prior to use. The biological sample can comprise serum and depletion can be of a common serum protein, such as albumin. Other common proteins that can be depleted include immunoglobulins (such as IgG, IgA, IgD and IgM), fibrinogens, Apolipoproteins (such as apolipoproteins A1, A2, and B), Transferrin, Prealbumin, Haptoglobulin, Plasminogen, Acid-1-Glycoprotein, Ceruloplasmin, Complement C3, Complement C1, Complement C4, alpha-2-macroglobins, and alpha-1-Antitrypsin.

The serum can be from a subject with or without a disease or condition, such as cancer. In one embodiment, the biological sample comprises a diseased tissue or tissue from a subject with a condition and a common serum protein, such as albumin is depleted.

Depletion can comprise filtration, fractionation, or affinity purification, or other methods known in the art. The biological sample can be diluted prior to being used to immunize an animal. Thus, the biological sample can be depleted of biological materials or contaminants, diluted, or both. Alternatively, the biological sample may not be depleted of biological materials or contaminants and/or diluted prior to being used to immunize an animal. Example of non-limiting methods and technologies that can be used for depletion of biological materials or contaminants include such techniques as electrophoresis (1D-PAGE, 2D-PAGE, capillary, free-flow, etc.), chromatography (reversed-phase, hydrophobic, ion exchange, size exclusion, affinity, etc), ultrafiltration, solvent precipitation, use of multiple affinity removal (MARS) columns for affinity mediated depletion of six high-abundant proteins (Agilent Technologies, Santa Clara, Calif., Part #5185-5984) including albumin, transferrin, haptoglobin, IgG, IgA, and alpha-1 antitrypsin, and other less common fractionation techniques. One classical strategy for albumin depletion that can be used involves the use of the hydrophobic dye Cibacron blue, a chlorotriazine dye which has high affinity for albumin, or other molecules, such as mimetic dyes or molecules which demonstrate greater specificity than Cibacron Blue. Other classical affinity mediums that can be used are Protein A, Protein G, and Protein A/G, which can be used for the depletion or removal of immunoglobulins, which represent the second most abundant proteins in the plasma or serum.

The first biological sample can be from a subject with a disease or condition and the second biological sample can be from a subject without the disease or condition, or vice-versa. For example, the first biological sample can comprise one or more disease or condition specific proteins, which the second biological sample can lack, or vice-versa.

The biological sample can be from a subject, such as directly from the subject or derived from cells from the subject. The subject can have a disease or condition. Alternatively, the subject may not have a disease or condition. For example, a first biological sample can be from a subject with a disease or condition and the second biological sample from a subject without the disease or condition. The subject can be non-responsive to a treatment or therapeutic. Alternatively, the subject can be responsive to a treatment or therapeutic. For example, a first biological sample can be from a subject that is non-responsive to a treatment or therapeutic and the second biological sample can be from a subject that is responsive to the treatment or therapeutic.

The first and second biological samples, and in some embodiments a third biological sample, can be from different subjects, sources, or cell lines. For example, a first biological sample can be from a first subject with a disease or condition and the second biological sample can be from a second subject without the disease or condition. A first biological sample can be from a first subject that is non-responsive to a treatment or therapeutic and the second biological sample can be from a second subject that is responsive to the treatment or therapeutic. The first biological sample can be from a subject before treatment and the second biological sample can be from a subject after treatment.

The first and second subject, can be in the same or different age or age group, be of the same or opposite sex, have the same or different race or ethnic background, have a similar or dissimilar lifestyle (such as diet, exercise, environmental conditions), or any combination thereof. For example, the first and second subject can be in the same age or age group, of the same sex, and have the same race or ethnic background, and similar lifestyle, with the first subject having a specific condition and the second subject may not having that specific condition. The first and second subject cane be in the same age or age group, of the same sex, and have the same race or ethnic background, but differ in their diet, and the first subject having a specific condition and the second subject not having the specific condition.

The first and second biological samples, and in some embodiments a third biological sample, can also be from the same subject, source, or cell line. For example, the first biological sample can from a subject at one timepoint and the second biological sample can be from the same subject at the same timepoint, or at an earlier or later timepoint. Biological samples can be taken from the same subject once or multiple times, such as at various timepoints or before or after various treatments. The later timepoint can be seconds, minutes, hours, days, weeks, months, or years after the first timepoint. More than one timepoint can be used, such as a first, second, third or more timepoints. For example, the first biological sample can be from a subject before treatment and the second biological sample can be from the same subject after treatment. Additional samples can be taken from the same subject, such as at one or more later timepoints, with or without additional treatments. In another embodiment, a first biological sample can be from a subject with a disease or condition at one timepoint, and the second biological sample can be from the same subject but at a later timepoint. Additional biological samples can be taken from the same subject. The biological samples can be taken when the subject is exhibiting certain symptoms of the disease or condition, or prior to exhibiting symptoms. The biological samples can be taken from a subject diagnosed with a disease or condition, or prior to being diagnosed with a disease or condition.

The biological sample can be a diseased sample, or a sample from a subject with a condition or disease. The biological sample can be a diseased tissue or cell, such as a breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer tissue or cell.

The biological sample can be from a subject with a disease or condition such as a cancer, inflammatory disease, immune disease, autoimmune disease, cardiovascular disease, neurological disease, infectious disease, metabolic disease, or a perinatal condition. For example, the disease or condition can be a tumor, neoplasm, or cancer. The cancer can be, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, or Burkitt's lymphoma. The disease or condition can also be a premalignant condition, such as Barrett's Esophagus. The disease or condition can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis. The disease or condition can also be a cardiovascular disease, such as atherosclerosis, congestive heart failure, vulnerable plaque, stroke, or ischemia. The cardiovascular disease or condition can be high blood pressure, stenosis, vessel occlusion or a thrombotic event. The disease or condition can also be a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The condition may also be fibromyalgia, chronic neuropathic pain, or peripheral neuropathic pain. The disease or condition may also be an infectious disease, such as a bacterial, viral or yeast infection. For example, the disease or condition may be Whipple's Disease, Prion Disease, cirrhosis, methicillin-resistant staphylococcus aureus, HIV, hepatitis, syphilis, meningitis, malaria, tuberculosis, or influenza. The disease or condition can also be a perinatal or pregnancy related condition (e.g. preeclampsia or preterm birth), or a metabolic disease or condition, such as a metabolic disease or condition associated with iron metabolism.

The biological sample can be administered to animal, wherein the administration can be by any means. At least two different biological samples can be used, in which each biological sample can be administered to the same or a separate animal. For example, a first animal can be administered a first biological sample and a second animal can be administered a second biological sample. Additional biological samples can be used, such as a third, fourth, fifth, or more biological sample can be administered to the same or to a third, fourth, fifth, or more animal.

The administration can be an immunization (for example, active or passive immunization) and can be performed by an infusion method, such as injection. For example, a first animal can be immunized with a first biological sample and the first or a second animal can be immunized with a second biological sample. Additional biological samples can be used to immunize the same or additional animals, such as a third, fourth, fifth, or more biological sample can be used to immunize the same or a third, fourth, fifth, or more animal. The method of injection or infusion can be intradermal, subcutaneous, intramuscular, intravenous, intraosseous, via foot pad, or intraperitoneal.

A single animal can also be administered additional biological samples. The additional biological samples can be the same or different as the initial or previous biological sample. For example, a first animal can be administered a first biological sample and a second animal can be administered a second biological sample. Both the first and second animals can then be given one or more additional biological samples, such that the first animal is given a second dose of the first biological sample and the second animal is given a second dose of the second biological sample. Additional doses can also be given, such that a second, third, fourth, fifth or more is given. The first or second dose can be the same or a different amount than each other or than one or more of the additional doses. The additional biological samples can be “boosts.” For example, a first animal can be immunized with a first biological sample and a second animal can be immunized with a second biological sample. Both the first and second animals can then be given boosts, such that the first animal is given a second dose of the first biological sample and the second animal is given a second dose of the second biological sample. Additional boosts can also be given, such that a second, third, fourth, fifth or more boosts are given.

The first biological sample can be from a subject with a disease or condition and the second biological sample, and in some embodiments the third biological sample, can be the control, such as from a subject without the disease or condition. The biological samples can also differ by being from different stages of a disease or condition from the same or different subject. The biological samples can also differ by being from different time points from the same or different subject, such as before or after treatment.

In some aspects, the first or second animals can be tolerized before administration of one or more biological samples. Subtractive immunization can be utilized to tolerize an animal. Subtractive immunization utilizes an immune tolerization approach that can enhance the generation of antibodies to desired antigens. For example, tolerization of both neonatal and adult mice against common, non-biomarker antigens (i.e., controls) prior to immunization can provide the means to recover one or more antibodies with a desired and/or defined specificity. The approach is based on tolerizing the host animal to immunodominant or otherwise undesired antigen(s) (i.e., a tolerogen) that may be structurally or functionally related to an antigen of interest. Tolerization of the host animal can be achieved by high zone tolerization, low zone tolerization neonatal tolerization, adult tolerization, drug-induced tolerization (for example, chemical immunosuppression with cyclophosphamide), or any combination thereof. For example, tolerance can be induced by exposing an animal to an antigen at an early stage of life, such as prior to maturation of the immune system, or, in adults, by exposing the animal to repeated low doses of a weak protein antigen (low-zone tolerance), or to a large amount of an antigen (high-zone tolerance).

The tolerized animal can then be inoculated with the desired biological sample or antigen and one or more of the antibodies generated by the subsequent immune response can be screened for the desired antigenic reactivity using any of the methods described herein. As a non-limiting example, a control biological sample, such as from a subject without a disease or condition, can be administered to an animal that will be immunized with a biological sample from a subject with the disease or condition. In some embodiments, the animal can be administered the control biological sample when the animal is immature or neonatal. These methods can augment the possibilities of the animal reacting to the biomarkers specific to a biological sample, for example a biological sample from a subject with a disease or condition, at the time of immunization with the biological sample. In some embodiments, a control biological sample can be one or more purified proteins, wherein the one or more purified proteins are non-biomarkers. In some embodiments, a control biological sample can be a biological sample from a subject without a disease or condition. In some embodiments, animals can be tolerized against multiple common cell types within a tissue homogenate and can then be immunized with one or more other tissue homogenates containing an additional unique cell population. Tolerogen administration can be oral, intravenous, intraperitoneal, intradermal, subcutaneous, intramuscular, intraosseous, or via foot pad.

Tolerance can be induced in an animal by introducing a first biological sample comprising one or more or a plurality of antigens from a desired source (such as a human or other animal tissue sample) to provide tolerance to the “background” antigens. At a later time (e.g., after maturation of the animal's immune system) the animal can be challenged with a second population of antigens. A second population of antigens can encompass all, or one or more, but not necessarily all, of the antigens of the first population (i.e., the “background” antigens). Additionally, a second biological sample comprising one or more or a plurality of antigens can comprise additional antigens not present in the first population. It is expected that a normal immune response (e.g., antibodies) to those antigens present in the second biological sample, but not in the first biological sample will be developed in the test animal. In this manner, development of antibodies specific to desired antigens for analysis (e.g., biomarkers for a disease, transcription factors, or any other immunogen) can be enhanced and background can be reduced.

As a nonlimiting example of such an approach, a neonatal test animal (e.g., a mouse) is administered a biological sample from a normal (i.e., non-diseased) human. Introduction of the tissue sample induces tolerance to the set of antigens present in the biological sample (or a sub-set thereof). After maturation of the test animal's immune system, a diseased tissue sample (e.g., a tumor sample, an infected lesion, etc.) is introduced into the animal. Antibodies produced by the test animal following challenge with the diseased tissue sample would be expected to be specific for biomarkers for the diseased sample (e.g., altered proteins, mutated proteins, newly produced proteins, etc.). Such antibodies can be analyzed via any of the methods disclosed herein and can also be used to construct arrays of antibodies specific for a particular disease state or other condition.

As another nonlimiting example, a first biological sample is administered to a first animal and a second animal, wherein the first biological sample comprises a first plurality of antigens. Subsequently, a second biological sample is administered to the first animal wherein the second biological sample comprises a second plurality of antigens. A third biological sample is administered to the second animal after administering the first biological sample, wherein the third biological sample comprises a third plurality of antigens. The third plurality of antigens comprises one or more additional antigens not present in the second plurality of antigens.

The methods described herein can further comprise comparing an immune response from a first non-human animal to an immune response from a second non-human animal, isolating an antibody-generating cell from a non-human animal for producing an antibody, isolating antibody-generating cells from a second non-human animal for producing antibodies with specificity to different biomarkers, identifying one or more or a plurality of biomarkers from a difference in the immune response from a first non-human animal to the immune response from a second human animal, screening an immune response from a second non-human animal using an array of proteome, or any combination thereof. Thus, the methods described above can be used to produce antibodies, produce antibodies with specificity to different biomarkers, identify one or more biomarkers, or profile a protein composition.

Any two, or three, or more pluralities of antigens used can be compared to each other, for example a first plurality of antigens can be compared to a second plurality of antigens, a first plurality of antigens can be compared to a third plurality of antigens, a second plurality of antigens can be compared to a third plurality of antigens, a third plurality of antigens can be compared to a fourth plurality of antigens, a second plurality of antigens can be compared to a first and third plurality of antigens, or any combination thereof. Any plurality of antigens can be the same as any other plurality of antibodies, for example, a first plurality of antigens and a second plurality of antigens can be the same, or a first plurality of antigens and a second plurality of antigens can be the same plurality of antigens, such as from the same biological sample. Any plurality of antigens can be the derived from the same source as any other plurality of antibodies, for example, a first plurality of antigens and a second plurality of antigens can be derived from the same source, for example, a first plurality of antigens and a second plurality of antigens can be derived from the same species, organism, biological sample, or purified biological sample. Any plurality of antigens can substantially overlap with any other plurality of antibodies, for example, a first plurality of antigens and a second plurality of antigens can substantially overlap, for example, a first plurality of antigens can comprise 10%, 15%,20%, 25%, 30%, 35%, 40%, 45%, 50%, 55%, 60%, 65%, 70%, 75%, 80%, 85%, 90%, 95%, 96%, 97%, 98%, 99%, or 100% of the antigens in a second plurality of antigens. Any plurality of antigens can comprise one or more of the antigens in any other plurality of antibodies, for example, a first plurality of antigens can comprise one or more of the antigens in a second plurality of antigens, for example, a first plurality of antigens can comprise 2, 3, 4, 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 or more of the antigens in a second plurality of antigens.

While the discussion above relates a first and second plurality of antigens, one skilled in the art would understand that any two, or three, or, four, or more pluralities of antibodies can be related or compared, be the same or not the same, be derived from the same source or not derived from the same source, or can substantially overlap or not substantially overlap.

Any plurality of antigens can comprise 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the antigens in any other plurality of antigens. Any plurality of antigens can comprise one or more of the antigens not in any other plurality of antigens, for example, a plurality of antigens can comprise 2, 3, 4, 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 or more of the antigens in a not in any another plurality of antigens. Any plurality of antigens can comprise 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the antigens in any other plurality of antigens and can comprise at least 2, 3, 4, 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 1000 or more of the antigens not in the any other plurality of antigens.

A first biological sample can be administered prior to maturation of the immune system, during maturation of the immune system, or after maturation of the immune system in a first non-human animal, a second non-human animal or both.

The immune responses from the animals administered the same or different biological samples can be compared to identify any differences. An animal administered a first biological sample can have a different immune response than an animal administered a second biological sample. The immune response from an animal immunized with a first biological sample can have a different immune response than an animal immunized with a second biological sample, and in some embodiments a third biological sample. The immune response can comprise a humoral immune response. A biological sample, such as serum, of each animal can be analyzed to determine any differences in the humoral immune response of one animal as compared to another. The supernatants from lymph node suspensions, spleen cell suspensions, or a combination thereof, from each animal can be analyzed to determine any differences in the humoral immune response of one animal as compared to another, for example, when immunizations are performed locally, such as into footpads. Lymphoid cells can be isolated from lymph nodes or from a spleen, for example, after localized injection to areas drained by the lymph node or systemic injections drained by the spleen.

Differences in the humoral immune responses of the animals administered or immunized with different biological samples can be determined by detecting the level of a humoral response of each animal to an epitope or antigen. For example, proliferation of memory lymphocyte subsets can be determined and can be a measure of the behavior of the immune cells following antigen exposure. This can be accomplished, for example, by flow cytometry using vital dyes, or by incorporation of detectable nucleic acid analogues, such as bromodeoxyuridine (BrdU). As a non-limiting example, tritiated thymidine (³[H]-thymidine) incorporation with subsequent detection by a scintillation counter can be used to measure antigen-driven proliferation. As a non-limiting example, identification of proliferation of lymphocyte subsets can be possible by staining subsets and activation markers such as CD2, CD3, CD4, CD8, CD21, MHC I and II, and CD25 with detection of incorporated BrdU. Another method of monitoring immune responses can be cytokine profiling. This method offers a qualitative feature that can be used to characterize a response as predominantly humoral (TH2) or cell mediated (TH1). Upregulation of cytokine message (mRNA) can be determined using real-time reverse transcriptase polymerase chain reaction (RT-PCR) to determine the relative quantity of specific cytokine mRNA relative to one or more other genes, such as housekeeping genes. For example, cytokine expression can be determined by flow cytometry using intracellular staining with anti-cytokine antibodies, which can be conjugated to fluorochromes to measure the frequency of cells making cytokine protein. The amount, concentration, or relative frequency of cytokines or cytokine expressing cells can also be measured using an immunoassay such as an ELISpot, ELISA, or FluoroSpot assay. For example, secreted cytokines can be quantified by ELISA using tissue culture supernatant of hybridomas, spleen cells, or activated lymphocytes, or by bioassay using responsive cell lines. Antigen-specific effector cell activity can also be assayed. One such method entails loading autologous target cells with a radiolabeled moiety, such as radiolabeled chromium (⁵¹Cr), and antigen, and determining the relative level of the radiolabeled moiety's release as a consequence of lysis by cytoxic T cells (CTL). Assays measuring lactate dehydrogenase (LDH) release can be substituted for such assays. Total cytoxicity or CTL-mediated cytotoxicity can be measured and used to assess the cell-mediated immune response. Serum neutralization (SN) or virus neutralization (VN) assays, or other methods known in the art can also be used to measure induction of systemic antigen-specific antibody production. Various cell lines and viral challenge strains at various concentrations can be used to conduct such assays.

A humoral response to an antigen of first animal administered a first biological sample can be the same, higher, or lower than a humoral response to the antigen of second animal administered a second biological sample, and in some embodiments a third biological sample. The humoral response to an antigen of a first animal immunized with a first biological sample can be the same, decreased, or increased as compared to a humoral response to the antigen of a second animal immunized with a second biological sample. A difference in humoral response can be qualitative or quantitative. In some embodiments, the difference can be statistically significant, such as by determined by a p value, for example, less than or approximately equal to 0.05, 0.04, 0.03, 0.02, 0.01, 0.009, 0.008, 0.007, 0.006, 0.005, 0.004, 0.003, 0.002, or 0.001 from a parametric analysis (e.g., Student's T test or Welch's T test) or by a non-parametric analysis (e.g., Wilcoxon-Mann-Whitney test or Kruskal-Wallis test), or other test.

A humoral immune responses from animals administered one or more different biological samples can be compared by determining the amount of antibody binding from each humoral immune response to one or more epitopes or antigens or any array thereof. For example, sera from animals immunized with different biological samples can be compared by detecting the level of antibody binding to an antigen. The amount of antigen binding from antibodies in the sera of a first animal immunized with a first biological sample can be the same, higher, or lower than the amount of antigen binding from antibodies in the sera of a second animal immunized with a second biological sample, and in some embodiments a third biological sample. A difference in antigen binding can be qualitative or quantitative. A difference can be statistically significant, such as by determined by a p value, for example, 0.05, 0.04, 0.03, 0.02, 0.01, 0.009, 0.008, 0.007, 0.006, 0.005, 0.004, 0.003, 0.002, or 0.001 from a parametric analysis (e.g., Student's T test or Welch's T test) or by a non-parametric analysis (e.g., Wilcoxon-Mann-Whitney test or Kruskal-Wallis test), or other test.

An antigen can be a protein or a peptide, a glycoprotein, a lipid, a glycolipid, a phospholipid, a complex sugar or a nucleic acid. An antigen can be attached or linked to a substrate, such as an array or particle. A library of antigens, such as a library of proteins or peptides, can be used to screen detect the humoral immune response of an animal. A library can comprise the proteome of a species, or a portion of the proteome of a species, such as at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the proteome of a species. A proteome can be of any organism, such as a bacterium, fungus, plant or animal. A proteome can be of a non-human animal. A non-human animal can be mammal, bovine, canine, equine, feline, ovine, porcine, or primate animal. For example, an animal can be a mouse, rat, rabbit, cat, dog monkey, or goat. A proteome can be that of a virus, bacterium, mycoplasma, parasite, fungus, or plant. A proteome can be the human proteome. A library can also comprise subsets of proteins of a particular class or proteins that share a common attribute, such as proteins that perform similar function, such as transcription factors, are in a particular signaling pathway, or involved in the development of specific disease processes. In one embodiment, an array comprising a proteome array comprising epitopes or antigens from a subject in which the biological sample was obtained can be used. An array can be used to decipher an immune response to generate data that lead to the identification of one or more unique antigens that are expressed in the biological sample. An array can be used to identification one or more specific antibodies, such as mMAbs.

A library of antigens can be present on an array and used to detect the level of humoral immune response, or antibody binding, of an animal. The humoral immune responses from animals administered different biological samples can be compared, and any difference in binding (such as presence, absence, increase, or decrease) to an antigen, such as a protein or peptide, can be used to identify that protein or peptide as a biomarker. For example, the amount of antigen binding from sera (for example, the amount of antigen binding of an antibody present in the sera) of a first animal administered a biological sample from a subject with cancer can be determined with a microarray of proteins, such as an array comprising all or part of a human proteome. The amount of antigen binding from sera of a second animal administered a biological sample from a subject without cancer can also be determined with a microarray of proteins. The two microarray analysis results can be compared to determine any differences in binding. A protein bound or detected by the sera from the first animal, but not by the sera from the second animal, can be identified as a biomarker for cancer. The animals may have been immunized according to any of the methods described herein, such as with the biological samples, or have been boosted with additional administrations of the biological samples prior to determining the antigen binding properties of the sera.

The amount of antigen binding from sera of a first animal administered a biological sample from a subject non-responsive to a therapeutic can be determined with a microarray of proteins, such as an array comprising all or part of a human proteome. The amount of antigen binding from sera of a second animal administered a biological sample from a subject that is responsive to the therapeutic can also be determined with a microarray of proteins. The two microarray analysis results can be compared to determine any differences in binding. A protein bound or detected by the sera from the first animal, but not by the sera from the second animal, can be identified as a biomarker for non-responsiveness to the therapeutic. A protein not bound or detected by the sera from the first animal, but bound or detected by the sera from the second animal can be identified as a biomarker for responsiveness to the therapeutic. The animals may have been immunized with the biological samples or have been boosted with additional administrations of the biological samples prior to determining the antigen binding properties of the sera.

The amount of antigen binding from sera of a first animal administered a biological sample from a subject with an advanced stage of a disease, can be determined with a microarray, such as an array comprising the human proteome. The amount of antigen binding from sera of a second animal administered a biological sample from a subject that is at an earlier stage of a disease can also be determined with a microarray of proteins. The two microarray analysis results can be compared to determine any differences in binding. A protein bound or detected by the sera from the first animal, but not by the sera from the second animal, can be identified as a biomarker for the advanced stage of the disease. The binding by the sera of the first and second animals can be compared to that of a third animal, which is administered a biological sample from a subject without the disease. A protein can be bound by the sera from both the first and second animals, but not the third, and there can be increased binding to the protein by the sera from the first animal as compared to the sera from the second animal. This protein can be identified as a biomarker for disease, in which the level can be an indicator of the stage or progression of the disease. The animals may have been immunized with the biological samples or may have been boosted with additional administrations of the biological samples prior to determining the antigen binding properties of the sera.

The amount of antigen binding from sera of a first animal administered a biological sample from a subject with a disease and without treatment can be determined with a microarray of proteins, such as an array comprising the human proteome. The amount of antigen binding from sera of a second animal administered a biological sample from the same subject, but taken after successful treatment, is also determined with a microarray of proteins. The two microarray analysis results can be compared to determine any differences in binding. A protein bound or detected by the sera from the first animal, but not by the sera from the second animal, can be identified as a biomarker for responsiveness to the treatment for the disease. The animals may have been immunized with the biological samples or have been boosted with additional administrations of the biological samples prior to determining the antigen binding properties of the sera.

Antibody binding can be detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

Antibody binding can be detected by detecting a label on the primary antibody (i.e. the antibody present in the sera of the animal). Alternatively, the primary antibody can be detected by detecting binding of a secondary antibody or reagent to the primary antibody. For example, the secondary antibody can be labeled. In some embodiments, an automated detection assay or high-throughput system can be utilized. For example, a protein array can be used.

Imaging Surface Plasmon Resonance Spectroscopy (SPR), Imaging Optical Ellipsometry (OE), or Reflectometric Interference Spectroscopy (RIFS) can be used to detect antibody binding. Antibody binding can be detected using an oblique-incidence reflectivity difference (OI-RD) technique, such as with a scanning microscope. Other label-free, real-time methods can detect cell surface antigen binding using unlabeled antibodies.

Data from the immune response of an animal can be stored on a computer system and used in future analyses. For example, the amount of antigen binding from sera of a first animal administered a biological sample from a subject with a disease and without treatment can be determined with a microarray of proteins, such as an array comprising the human proteome. The data from this first animal can be stored and compared to the data from another animal administered a different biological sample.

A reagent to a biomarker identified by a method disclosed herein can also be generated using the animals that produced the humoral immune response to identify the biomarker. An antibody can be produced by a method comprising administering to a first animal a first biological sample and comparing an immune response from the first animal to an immune response from a second animal and identifying one or more biomarkers from a difference in the immune response from the first animal to the immune response from the second animal, as described above. The second animal may have been administered a second biological sample and the administration can be an immunization. Thus, a reagent to a biomarker identified by a method disclosed herein can be subsequently or sequentially generated using the animals that produced the humoral immune response to identify the biomarker.

A first animal administered a first biological sample can then be further administered a plurality of the identified biomarkers. The administration of the plurality of the identified biomarkers can be an immunization of the animal against the biomarker. An antibody-generating cell from the animal can then be isolated for producing an antibody to the biomarker. A plurality of antibodies with specificity to different biomarkers can be produced. The method can comprise administering to a first animal a first biological sample and comparing an immune response from the first animal to an immune response from a second animal and identifying a plurality of biomarkers from a difference in the immune response from the first animal to the immune response from the second animal, as described above. The second animal may have been administered a second biological sample and the administration can be an immunization. The first animal administered the first biological sample can then be further administered the plurality of identified biomarkers. The administration of the identified plurality of biomarkers can be an immunization of the animal against the plurality of biomarkers. Antibody-generating cells from the animal can then be isolated for producing antibodies to the plurality of biomarkers.

The biomarker administered to an animal can be a protein, peptide, lipid, or nucleic acid, such as RNA or DNA, or fragment thereof. An antibody-generating cell from the animal can then be isolated for producing an antibody to the biomarker. A plurality of different biomarkers can be administered to the animal, for example different proteins. A plurality of different types of biomarkers (for example, a combination of DNA and proteins of the different or same biomarker) can be administered to the animal. A plurality of antibody-generating cell from the animal can then be isolated for producing a plurality of antibodies to the different biomarkers. The antibody-generating cell can be used to generate a hybridoma. The antibody-generating cell can be a B-cell. The B-cell can be fused to a cell, such as a myeloma cell, to create a hybridoma. An antibody to the biomarker can then be produced and isolated from the hybridoma. The antibody can be a polyclonal or monoclonal antibody.

As the animal given the biomarker had previously been administered, or immunized, with a biological sample comprising the biomarker, the animal can produce antibodies to the biomarker more quickly or with a higher yield as compared to an animal that had not been previously administered a biological sample comprising the biomarker. The antibody against the biomarker can already be present in the animal, as established by identification of the biomarker, such as by protein array analysis as described herein. Immune cells against the biomarker can also already be present in the animal.

Any suitable method may be used to generate the antibodies disclosed herein. For example, a biomarker composition, comprising the identified biomarker or a plurality of identified biomarkers, can be produced in vitro, such as by any recombinant methods known in the arts. The biomarker composition can further comprise a suitable carrier or diluent and can be administered to the animal under conditions that permit the production of antibodies. For enhancing the antibody production capability of the animal, complete or incomplete Freund's adjuvant can also be administered. The biomarker composition can be administered once a day or one or more times a week, such as one a week, twice a week, thrice a week, four times a week, five times a week, six times a week, or seven times a week, or every 2 to 4 weeks, such as every 2 weeks, 3 weeks, 4 weeks, 5 weeks, 6 weeks, 7 weeks, 8 weeks, or more. The biomarker composition can be administered once, or a total of about 2 times to about 10 times. The biomarker composition can be administered 2, 3, 4, 5, 6, 7, 8, 9, 10 or more times. Administration can be by any method known in the art, such as, but not limited to, administration subcutaneously, intraperitonealy, intravenously, via foot pad, and the like.

For preparing monoclonal antibody-producing cells, an individual animal whose antibody titer has been confirmed can be selected, and after the final immunization, such as from 2 to 5 days after, its spleen or lymph node can be harvested and antibody-producing cells contained therein can be fused with myeloma cells to prepare the desired monoclonal antibody producer hybridoma.

Hybridomas can be generated by fusing two cell types, for example, immune B cells and a culture-stable myeloma cell line. This can be carried out in the presence of a fusogenic compound such as PEG. The desired hybrid cell products can be selected from among the unfused cells by taking advantage of the presence of two metabolic routes of pyrimidine/purine synthesis, the de novo and scavenging pathways. The myeloma cell lines commonly used are deficient in the salvage pathway, as they have been selected for resistance to 8-azaguanine or 6-thioguanine and are thus hypoxanthine-guanine phosphoribosyl transferase (HGPRT) deficient. Without the salvage pathway for viability, these cells require the de novo pathway, which, however, can be blocked with aminopterin. B cell myeloma hybrids can grow in the presence of aminopterin because the immune B cell donates a wild-type HPRT enzyme that supports processing of scavenged hypoxanthine (H) and thymidine (T). Fusion reactions can thus be plated in HAT medium to eliminate unfused immune cells and myeloma cells but can be permissive for hybridoma outgrowth. The most useful myeloma cell lines are those such as X63-Ag8.653, NSW and Sp2/0, Ag-14, which do not secrete their own immunoglobulin heavy or light chains that would contaminate the product contributed by the B cell. Examples of myeloma cells include, but are not limited to, NS-1, P3U1, SP2/0, AP-1 and the like cells. The cell fusion can be carried out according to known methods.

Measurement of the antibody titer in antiserum can be carried out, for example, by reacting the labeled protein and antiserum and then measuring the activity of the labeling agent bound to the antibody. The proportion of the number of antibody producer cells (spleen cells) and the number of myeloma cells to be used can be optimized and performed by methods known in the art. The proportion of the number of antibody producer cells (spleen cells) and the number of myeloma cells to be used can be about 1:1 to about 20:1, for example about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1 ratio can be used. PEG, such as PEG 1000-PEG 6000 can be added in a concentration of about 10% to about 80%, for example 10%, 15%, 20%, 25%, 30%, 35%, 40%, 45%, 50%, 55%, 60%, 65%, 70%, 75%, or 80%. Cell fusion can be carried out efficiently by incubating a mixture of both cells at about 20° C. to about 40° C., such as about 30° C. to about 37° C. for about 1 minute to 10 minutes. For example, a mixture of both cells can be can be incubated at about 20° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. for about 1, 2, 3, 4, 5, 6, 7, 8, 9, or 10 minutes.

Various methods can be used for screening for a hybridoma producing the antibody against the biomarker as known in the arts. For example, a supernatant of the hybridoma can be added to a solid phase (e.g., microplate) to which antibody is adsorbed directly or together with a carrier and then an anti-immunoglobulin antibody (for example, if mouse cells are used in cell fusion, anti-mouse immunoglobulin antibody is used) or Protein A or Protein G labeled with a radioactive substance or an enzyme can be added to detect a monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect a monoclonal antibody against the protein bound to the solid phase.

Selection of a monoclonal antibody can be carried out according to any known method or its modification. A medium for animal cells to which HAT (hypoxanthine, aminopterin, thymidine) are added can be employed. Any selection and growth medium can be employed as long as the hybridoma can grow. For example, RPMI 1640 medium or GIT medium containing about 1% to 20%, such as 1%, 2%, 3%, 4%, 5%, 6%, 7%, 8%, 9%, 10%, 11%, 12%, 13%, 14%, 15%, 16%, 17%, 18%, 19%, or 20% fetal bovine or fetal calf serum, a serum free medium for cultivation of a hybridoma (SFM-101, Nissui Seiyaku), and the like can be used. The cultivation can be carried out at 20° C. to 40° C., such as about 20° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. for about 5 days to 3 weeks, such as about 5 days, 6 days, 1 week, 2 weeks, or three weeks under about 1-10% CO₂ gas, such as about 1%, 2%, 3%, 4%, 5%, 6%, 7%, 8%, 9%, or 10% CO₂ gas. An antibody titer of the supernatant of a hybridoma culture can be measured according to the same manner as described above with respect to the antibody titer of the anti-protein in the antiserum.

Separation and purification of a monoclonal antibody to a biomarker can be carried out according to the same manner as those of conventional polyclonal antibodies, such as separation and purification of immunoglobulins, ((for example, salting-out, alcoholic precipitation, isoelectric point precipitation, electrophoresis, adsorption and desorption with ion exchangers) (e.g., DEAE)), ultracentrifugation, gel filtration, or a specific purification method wherein an antibody is collected with an active adsorbent such as an antigen-binding solid phase, Protein A, or Protein G, and dissociating the binding to obtain an antibody.

Polyclonal antibodies may be prepared by any known method or modifications of these methods. For example, a biomarker composition comprising a biomarker and a carrier protein can be prepared and the animal can be immunized by the biomarker composition as described. A material containing the antibody against the biomarker can be recovered from the immunized animal and the antibody can be separated and purified.

Any carrier protein and any mixing proportion of the carrier and a hapten can be employed. The hapten can be cross-linked on the carrier and used for immunization. For example, bovine serum albumin, bovine cycloglobulin, keyhole limpet hemocyanin, etc. may be coupled to a hapten in a weight ratio of about 0.1 parts to about 20 parts, or about 1 part to about 5 parts per 1 part of the hapten, such as about 0.1 parts, 0.2 parts, 0.3 parts, 0.4 parts, 0.5 parts, 0.6 parts, 0.7 parts, 0.8 parts, 0.9 parts, 1 part, 2 parts, 3 parts, 4 parts, 5 parts, 6 parts, 7 parts, 8 parts, 9 parts, 10 parts, 11 parts, 12 parts, 13 parts, 14 parts, 15 parts, 16 parts, 17 parts, 18 parts, 19 parts, or 20 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like, can be used. The condensation product or together with a suitable carrier or diluent can be administered to a site of an animal that permits the antibody production. For enhancing the antibody production capability of the animal, complete or incomplete Freund's adjuvant can also be administered. The biomarker composition can be administered once a day or one or more times a week, such as one a week, twice a week, thrice a week, four times a week, five times a week, six times a week, or seven times a week, or every 2 to 4 weeks, such as every 2 weeks, 3 weeks, 4 weeks, 5 weeks, 6 weeks, 7 weeks, eight weeks, or more. The biomarker composition can be administered once, or a total of about 2 times to about 10 times. The biomarker composition can be administered 2, 3, 4, 5, 6, 7, 8, 9, 10 or more times.

A polyclonal antibody can be recovered from blood, ascites and the like, of an animal immunized by the above method. The antibody titer in the antiserum can be measured according to the same manner as that described above with respect to other antibodies and the supernatant of the hybridoma culture. Separation and purification of the antibody can be carried out according to the same separation and purification method of immunoglobulin as that described with respect to the above monoclonal antibodies.

Antibody binding can be detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc., as described above.

Antibody binding can be detected by detecting a label on the primary antibody. Alternatively, the primary antibody can be detected by detecting binding of a secondary antibody or reagent to the primary antibody. For example, the secondary antibody can be labeled. An automated detection assay or high-throughput system can be utilized.

For example, in a capture micro-enzyme-linked immunosorbent assay (ELISA), an antibody/antigen reaction can be made measurable by immobilization of the antibody and subsequent direct or indirect colorimetric, fluorescent, luminescent or radioactive detection of bound, labeled antigens. For example, the antigen can be labeled by biotin or other labels, which will allow downstream detection.

Immobilized antibodies will generally bind to a single antigenic determinant present. The antigenic determinant can be labeled, such as through labeling of the biomarker comprising the antigenic determinant The specificity of this reaction will permit quantification in the ELISA measurements. The ELISA reaction can be used in a high throughput format to screen all hybridoma supernatants via the following steps. Screening assays built on other principles than an ELISA can be deployed (e.g., antibody microarrays, high-throughput screening based on MALDI/MS and/or multi-channel capillary electrophoresis). ELISA or microarray data can be evaluated, e.g., by published methods. The goal of the data analysis process is the selection of hybridoma supernatants that show the best collection with an important clinical parameter and can be specific to one of the analyte groups.

Antibodies, monoclonal or polyclonal, produced by the methods and systems disclosed herein can be subjected to specificity profiling. A library of antigens can be screened with the antibodies produced by a hybridoma. For example, the antigen can be a protein or a peptide, a glycoprotein, a lipid, a glycolipid, a phospholipid, a complex sugar or a nucleic acid. The library of antigens, such as a library of proteins or peptides, can be attached or linked to an array. The library of antigens, such as a library of proteins or peptides, can then be used for specificity profiling of the antibody, such as a monoclonal antibody against an identified biomarker. The library of antigens used for specificity profiling can be the same as the library used for initial identification of the biomarker.

Also provided herein is a library of reagents to the biomarkers identified by a method disclosed herein. In the library or reagents can comprise a plurality of antibodies. The antibodies can comprise monoclonal or polyclonal antibodies, or fragments or derivatives thereof. The library of antibodies can comprise a plurality of different antibodies, each having the same target (i.e. recognizing the same biomarker), but with differing specificity to the biomarker, such as having differing binding constants. Alternatively, the library of antibodies can comprise a plurality of antibodies, each antibody binding a different target or biomarker. The library can comprise at least about 5, 10, 50, 150, 200, 250, 300, 350, 400, 450, 500, 550, 600, 650, 700, 750, 800, 850, 900, 950, or 1000 different antibodies. The one or more antibodies can be attached to a substrate, such as an array or particle. Thus, a plurality of particles with antibodies attached or an antibody array can be produced. The plurality of particles or antibody array can be used to screen or detect biomarkers, such as biomarkers in a biological sample (for example, transcription factors). The sample can be a biological sample from a subject with or without a condition or disease, as described herein.

A reagent to a biomarker identified by a method disclosed herein can also be a therapeutic. For example, an antibody produced by a method disclosed herein can be used as a therapeutic. The reagent, such as an antibody or a fragment or derivative thereof, of the present disclosure can be formulated for administration to human and non-human animals, such as, but not limited to, a bovine, avian, canine, equine, feline, ovine, porcine, or primate animal. For example, the mammal can be a mouse, rat, rabbit, cat, dog monkey, or goat.

Pharmaceutical compositions of the reagent, such as an antibody or a fragment or derivative thereof, may comprise an effective amount of the reagent, for example, an amount that modulates expression of a biomarker, in admixture with a pharmaceutically acceptable carrier. Examples of pharmaceutically acceptable carriers or solutions include, but are not limited to, aluminum hydroxide, saline and phosphate buffered saline. The reagent admixture with a pharmaceutically acceptable carrier can be formulated with other components that modulate expression of the biomarker.

The reagent, such as an antibody or a fragment or derivative thereof, may be administered to a subject for the treatment of a condition or disease. Conventional pharmaceutical carriers, aqueous, powder or oily bases, thickeners and the like may be necessary or desirable. Formulations for parenteral administration may include sterile aqueous solutions which may also contain buffers, liposomes, diluents and other suitable additives. For intramuscular, intraperitoneal, subcutaneous and intravenous use, the reagent-containing formulation may generally be provided in sterile aqueous solutions or suspensions, buffered to an appropriate pH and isotonicity. Suitable aqueous vehicles include Ringer's solution and isotonic sodium chloride.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1—Work flow

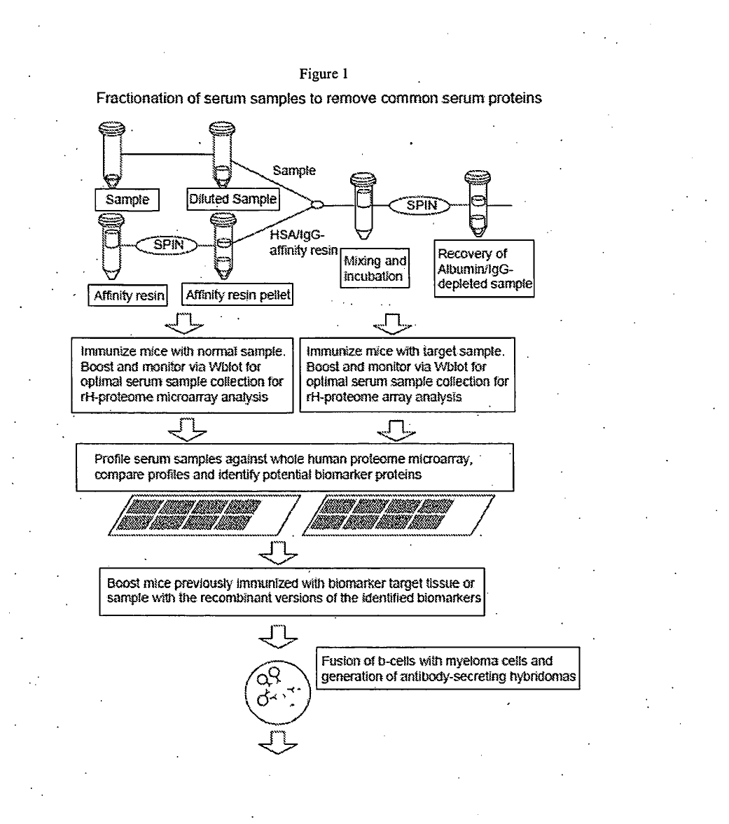

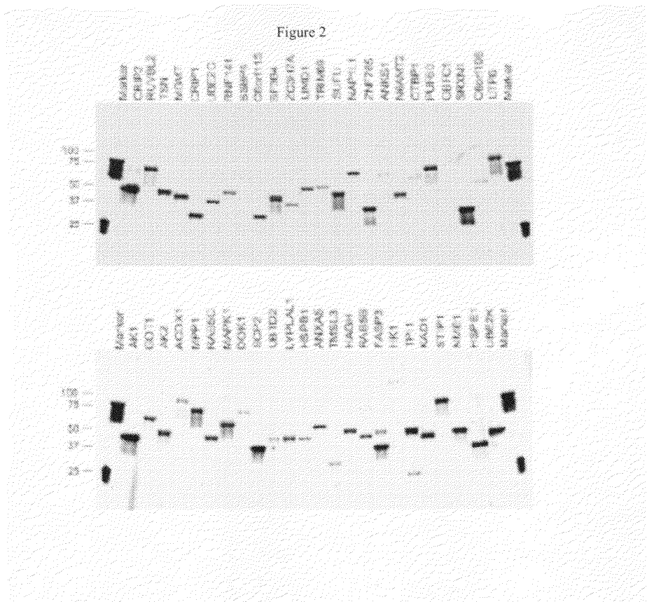

Sera from a cancer patient (“target sample”) and from a subject without cancer (“normal sample”) were obtained. The samples were each diluted and added to an HSA/IgG-affinity resin to deplete albumin and IgGs in the sera (see FIG. 1). One group of mice was immunized with the target sample (“target mice”) and another group of mice was immunized with the normal sample (“normal mice”). The target mice were given boosts of the target sample and the normal mice were given boosts of the normal sample. Sera from both groups of mice were monitored by Western blot analysis for optimal serum sample collection.

Serum samples were obtained from the target mice and from the normal mice. Each serum sample was subjected to profiling against a human proteome array. Antibodies in the serum samples were labeled and the serum samples were incubated with the array. The arrays were washed and detection of binding was performed. The binding profiles of the serum samples were compared and one of the proteins (“Protein X”) on the array was bound by the serum sample from the target mice but not by the serum sample from the normal mice. Recombinant Protein X was used to immunize the target mice. B-cells from the target mice were obtained and fused to myeloma cells to generate antibody-secreting hybridomas. Monoclonal antibodies produced by the hybridomas were then subjected to specificity profiling with a human proteome array.

### Example 2—Subcloning ˜17,000 Full-Length Human ORFs into an Expression Vectors

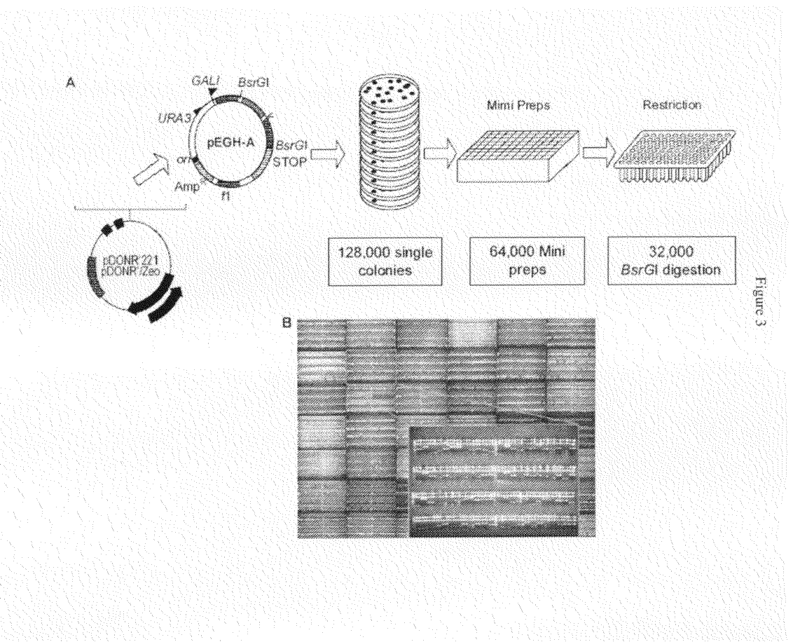

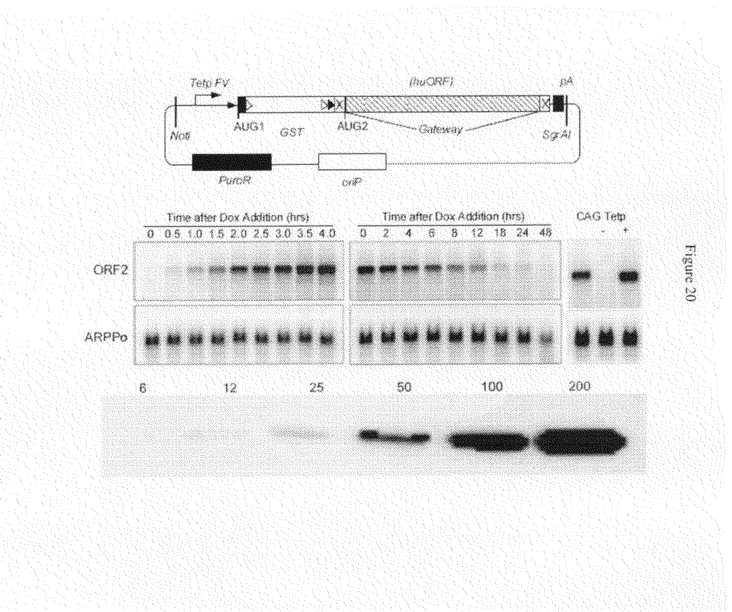

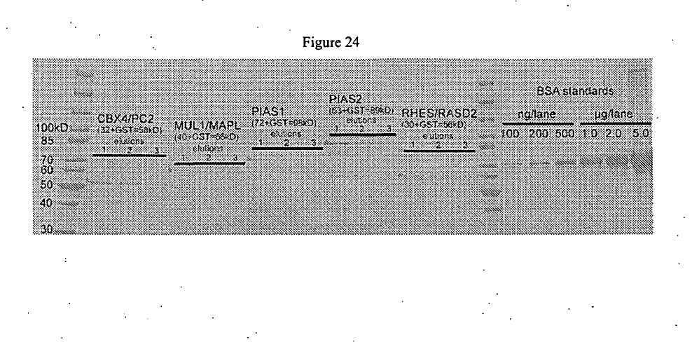

For highly efficient subcloning of libraries of human ORFs into a wide variety of destination vectors, all in frame, without the use of restriction enzymes, GATEWAY™ technology was utilized based on phage lambda integration proteins (FIG. 20). Invitrogen's Ultimate Human ORF™ collection representing more than 16,000 sequence validated human ORFs was cloned in the Gateway™ Entry vector, which allows for convenient subcloning the inserts into various Gateway™. Destination vectors were used for expression and functional analysis of the target protein in a variety of hosts, including E. coli (FIG. 24), yeast, baculovirus, CHO cells, and mammalian cell lines, as well as cell-free transcription and translation coupling systems. After attaining the library a complete human expression library was subcloned into a yeast expression vector, enabling construction of a near-complete Human Proteome Microarray (Hu-PM). In addition to the commercially available fully sequence verified human ORF collection of more than 16,000 unique ORFs, an additional 1,000 full-length human ORFs have been subcloned into the same Gateway entry vector by others. To increase the Hu-PM content from 17,000 proteins to 18,550, 1550 additional full-length Gateway-compatible ORF clones will be purchased from Thermo Fisher. A Gateway-compatible expression vector for yeast (pEGH-A) was constructed that, upon galactose induction, produces N-terminal 6x-His-GST fusion proteins in yeast (FIG. 3).

Subsequently, all human ORFs (about 17,000) were subcloned at a success rate of 99%, as confirmed by restriction digestion. All starting clones used to generate the yeast expression vectors from which the proteins are purified had their ORFs completely sequenced. Spot sequencing of 200 randomly selected yeast clones showed 100% correct assignment to wells i.e., providing very high confidence in collection quality. The 5′ junctions of that entire collection will be sequenced once the human expression library has been constructed as a validation step, Three replicates of the collection were prepared, from one of which the entry plasmid DNAs were extracted, and the quality of this plasmid DNA was determined on agarose gels. The resulting recombinants were then transformed into bacteria and single colonies were selected on Amp-containing LB-agar plates. For each recombination, four single colonies were picked to generate glycerol stocks, two of which were further processed to extract plasmid DNAs in a 96-well format. The extracted plasmid DNAs were digested with a restriction enzyme to release the inserts, and run on agarose gels to examine the vector and insert sizes as an indicator of successful subcloning (FIG. 3, right panel). Each restriction digest was scored based on expected insert sizes and the success rate was determined as 99.3%. Validation was performed on over 200 randomly selected LR clones by sequencing and 100% were correct. The confirmed LR constructs were rearrayed to generate a master set of expression clones for yeast. Similar large scale cloning was completed in a bacterial expression vector. This experiment will be repeated with a human expression vector, complete with 5′ junction sequencing of the entire human expression library.

### Example 3—Design and Fabrication of a 5000 Human Antigen Microarray

A pilot experiment was conducted to test the ability to rapidly purify correctly folded recombinant proteins for microarray production. These proteins fall into five different functional categories: transcription factors and transcription co-regulators, RNA binding proteins, protein kinases, chromatin-associated and chromatin-modifying proteins, and mitochondrial proteins. Proteins were placed in these categories on the basis of primary sequence, literature, and Gene Ontology annotation. The ORFs expressed represented as many as 85% (in the case of transcription factors) of all human proteins in the relevant functional category. Over 90% of expressed proteins were purified at sufficient levels for array construction. Several functional tests were performed to confirm that the expressed proteins were functional after being immobilized on solid surfaces, including but not limited to, autophosphorylation assays on a kinase chip containing 119 individually purified protein kinases from yeast and observed that approximately 85% of the kinases showed detectable kinase activity. Most of these protein kinases maintained their enzymatic activity. Another line of evidence came from recent studies on profiling the DNA binding activity of transcription factors using a protein chip approach. Using predicted DNA motifs, Snyder and colleagues demonstrated that specific interactions between various DNA motifs and transcription factors could be readily identified and profiled using a protein chip composed of about 300 yeast transcription factors. This approach was extended to the study of interactions between DNA motifs and human transcription factors. Using a pilot protein chip composed of about 1,000 human transcription factors, the known DNA motifs are shown to specifically bind to their documented transcription factors, and point mutations in these motifs dramatically reduced their binding affinity.

### Example 4—Fabrication of a Human Proteome Microarray (Hu-PM)

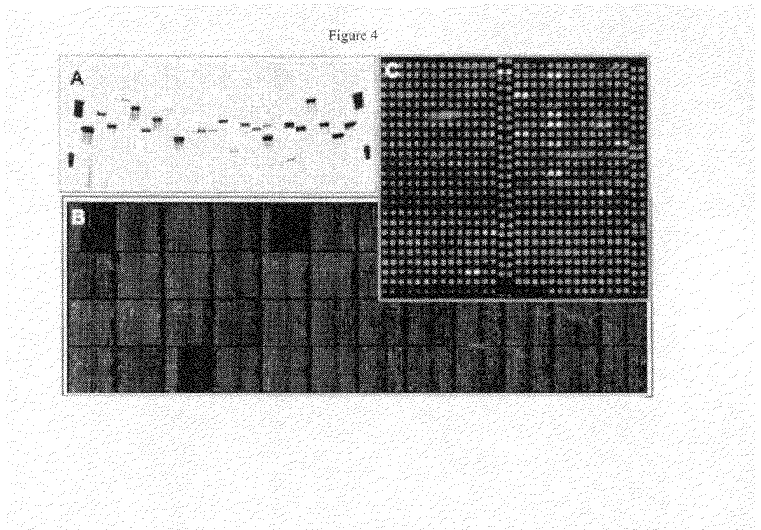

To purify the complete set of 17,000 human protein antigens from yeast cells, the entire master set of human ORFs cloned in pEGH-A were transformed into yeast, single colonies were picked, and glycerol stocks were prepared. The yeast cells were induced for recombinant protein production and stored at −80° C. To monitor the quality of induced cultures, 24 random strains were inoculated in duplicate for each batch of culture preparation and were processed through the protein purification step first. Using immunoblotting and silver staining, the success rate of culture induction was estimated (FIG. 4A). Success was indicated when at least 85% of the purified proteins showed a major band at the expected MW range in both immunoblot and silver staining analyses. Using this standard, all 17,000 antigen proteins were purified at a success rate of 85%. The purified human antigen proteins were spotted using a microarrayer onto various glass surfaces (e.g., FAST, Ni-NTA and FullMoon) to produce the Hu-PMs. The quality of the Hu-PMs was monitored by probing the slides with anti-GST antibodies and Cy3-labeled secondary antibodies (FIG. 4B). The Hu-PMs were scanned and signals acquired and analyzed using GenePix software. The results indicated that the Hu-PMs were of high quality and >90% of the printed proteins produced signals significantly higher than background (FIG. 4C).

### Example 5—Protein Microarray-Based Approach for High-Throughput mAb Generation in Mice

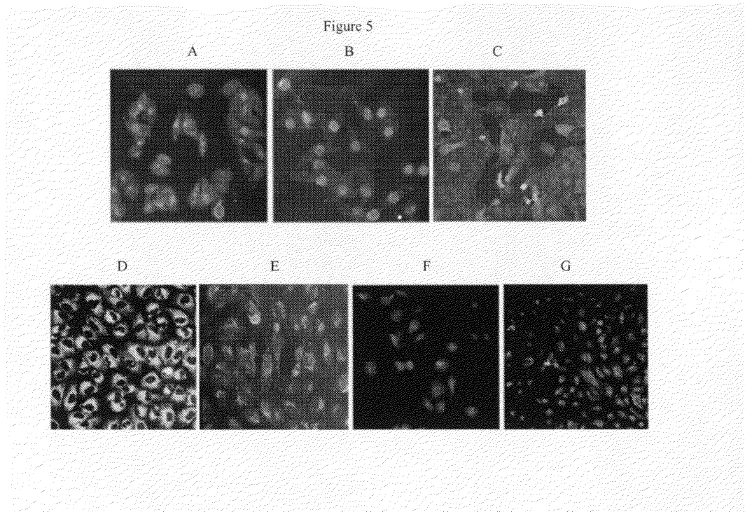

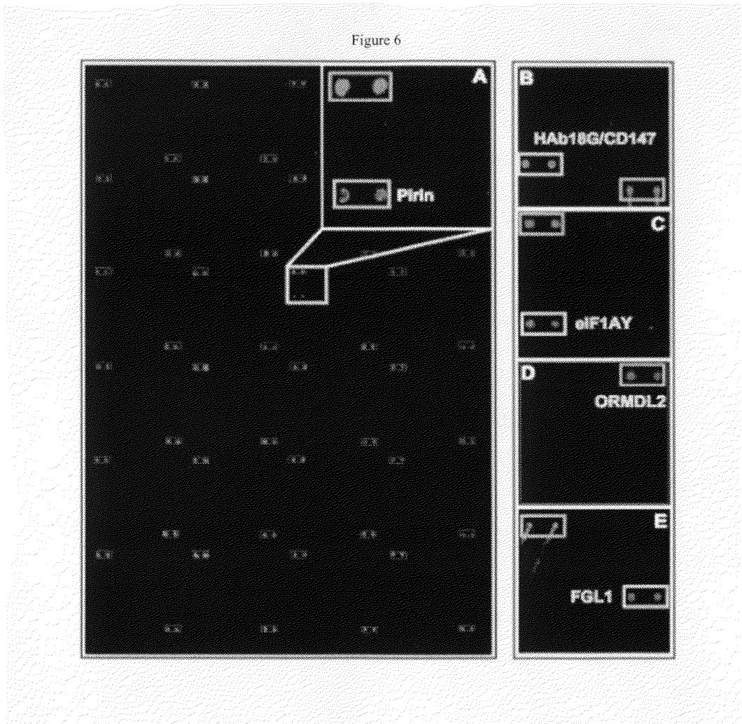

Others have previously demonstrated it was possible to generate mAbs in mice in a high-throughput fashion, developing a “shotgun” immunization method coupled with Protein Microarray technology to deconvolute the corresponding antigens. This was converted to a much more efficient Proteome Microarray based approach. In a Protein Microarray pilot study, live cells were injected from normal human liver into ten mice. After three immunizations, the murine spleen cells were fused with myeloma cells to generate 3,000 hybridomas. Seven human cell lines were then used to screen the hybridomas for binding activity. Fifty-four hybridomas were found to secrete mAbs that could recognize proteins in these cells (FIG. 5). SMMC-7721 cells were stained by mAbs 1G9, and 1E3, respectively. CCC-HEL-1 cells were stained by mAbs B4B5C5, and 2B2, respectively. SMMC-7721 and CCC-HEL-1 cells were stained by ascites from un-immunized mice as negative controls. Ascites samples of the 54 mAbs were prepared, and the antibody concentrations were determined to be in the range of 10-20 mg/mL. To identify their corresponding antigens, a human protein chip of 1,058 unique human liver proteins was constructed. To rapidly identify the corresponding antigens of the mAbs, 54 ascites were arrayed in a 7×8 format to create seven horizontal and eight vertical pools. The resulting 15 pools were separately incubated on the protein chips at a 70,000 or 80,000 fold dilution, and the bound mAbs were detected with Cy3-labeled antimouse IgGs. Using highly stringent criteria to deconvolute the results, five positive mAbs were identified. To reconfirm the results and determine the specificity of each positive mAb, the five mAbs were individually probed to the human protein microarrays via the above-described protocol, and confirmed to bind to liver proteins (FIG. 6), including Pirin, fibrinogen-like protein 1 (FGL1) precursor, ORM1-like 2 (ORMDL2), eukaryotic translation initiation factor 1A,Y (eIF1AY), and HAB18G/CD147 with high specificity. A full image of a human liver protein microarray that was probed with mAb 3A3b can be seen in FIG. 6A. For example, (FIG. 6A and inset), mAb 3A3b only recognizes a single protein, Pirin, with no obvious cross-reactivity to the other 1,057 unique proteins on the array. Comparable specificities were also observed for mAbs 1G9, 1E3, B4B5C5, and 2B2 against FGL1, ORMDL2, eIF1AY, and HAb18G/CD147, respectively (FIG. 6B-E).

### Example 6—Validation of Ab-Antigen Interactions

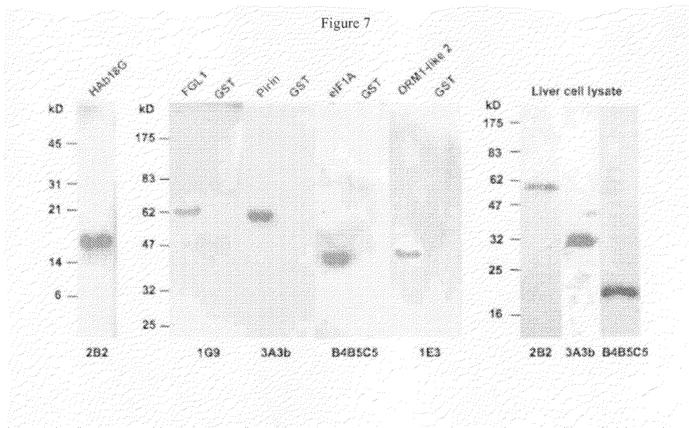

To produce high-quality mAbs and screen for their corresponding antigens in higher throughput, the predicted antibody-antigen interactions were validated by using traditional immunoblot analysis using the mAbs against the five purified recombinant proteins. As illustrated in FIG. 7, four GST fusion proteins of Pirin, FGL1, ORMDL2, and eIF1AY, as well as an 18-kD fragment of HAb18G/CD147, were recognized by their corresponding mAbs with no cross reactivity to GST Immunoblot analysis of human liver lysates was then applied using the five mAbs. Ascites 3A3b, B4B5C5, and 2B2 each recognized a single band at the respective positions of 32, 20 and 50 kD, in agreement with the expected molecular weights of full length Pirin and HAb18G/CD147, respectively (FIG. 7). The slight mobility reduction of the eIF1AY band (20 kD versus 16 kD) might be caused by either low resolution of the gel or protein posttranslational modifications such as phosphorylation and/or glycosylation in human liver cells. The other two mAbs did not show any significant signals in immunoblot analysis. It is conceivable that these two proteins are in low abundance in human liver tissues or that these antibodies may not recognize the denatured protein.

### Example 7—Generation and Characterization of Monospecific Monoclonal Antibodies (mMAbs)

Over 2,000 IgG secreting mAbs were generated in response to immunization with both recombinant human proteins and live human cancer cell lines. The more comprehensive microarray discussed above that contains 17,000 human proteins was used to analyze the binding specificity of 88 of these mAbs. To reduce the number of microarrays used for this analysis, equal mixtures of supernatants from up to seven different hybridomas were generated, and intersectional analysis was used to interrogate the specificity of these pooled antibodies. 11 of the 88 mAbs were determined to be truly monospecific, in that only a single protein on the microarray is recognized by these mAbs. Seven other mAbs bound to only three different proteins on the array, showing highly restricted specificity. For four of the monospecific mAbs, no commercially antibodies of any sort (polyclonal or monoclonal) are available, while it is not know whether any of the commercially available antibodies to the other seven are monospecific. Most of the human proteins can be expressed from a proprietary expression vector in E. coli (FIG. 24).

### Example 8—Antibody Production

Several methods of immunization have been described for inducing polyclonal antibody responses. The less inflammatory, but higher titer-inducing adjuvant mixtures of synthetic polymers of polyoxypropylene and ethylene with metabolizable oils, such as TiterMax (CytRx Corp.), can reduce the likelihood of deleterious inflammatory responses at sites of injection. Standard immunization schedules generally use an initial immunization, followed by repeated boosts at 2-3 week intervals until antibody titers are considered sufficiently high as determined by small volume bleedings. This lengthy immunization scheme can be shortened in at least two ways. Rapid immunizations at multiple sites (RIMMS) uses a single round of up to four dorsal subcutaneous injections of antigen, followed 14 days later by an intraperitoneal (IP) boost and 3-4 days later by spleen cell harvest. Another method of even shorter duration uses a single round of injection of antigen/adjuvant into the rear footpads, followed by harvesting of draining (popliteal and inguinal) lymph nodes 7-14 days later, without the need for a booster injection. This latter method has been shown to yield immune B cells producing antibodies that have undergone affinity maturation and IgM to IgG class switching as a result of the accelerated maturation of the immune response in the peripheral immune tissues versus the spleen.

In a standard scheme for making monoclonal antibodies, at least two types of screening can be done to identify the desired hybridomas. Typically, this is done by analyzing aliquots of each microtiter well culture supernatant from the dispersed cell fusion reaction. An ELISA type of assay, in which antigen is immobilized onto a 96 well polyvinyl plate and then incubated with supernatants from all of the colony-containing wells, can be devised for each individual antigen. The cells in the culture wells producing antigen-binding antibody are harvested and replated in diluted form for isolation of clonal colonies. This limiting dilution cloning step requires up to 2 weeks of incubation for individual cells to grow into assayable colonies.

Commonly built into the ELISA assay is the second type of needed screen, which reveals the class of antibody being secreted by the hybridoma. Of the five classes of immunoglobulin, IgG class antibodies are the most useful for research purposes. If the secondary antibody in the ELISA that tests for the presence of antibody recognizes only the IgG class, then only those clones producing this class will be identified. Thus the need to culture the less useful IgM secretors is eliminated. The two final steps in the isolation of monoclonal antibodies involve the establishment of the clonality of the hybridoma lines, and then the expansion of those lines to produce useful amounts of antibody. When the standard methods have been used to produce a hybridoma, the cell line needs to be cloned to eliminate the presence of contaminating hybridomas that may be producing unrelated antibodies. This is typically carried out by limiting dilution plating of the cells into microtiter wells, followed by a rescreening of the supernatant with an antigen-specific ELISA. The need for limiting dilution cloning can be bypassed if the original fusion reaction is plated in semi-solid medium (methylcellulose) in Petri dishes, since the cells grow out as defined clones from the beginning. Clones that are expressing antibodies to the antigen can be identified by including antigen in the semisolid medium, in effect carrying out an Ouchterlony reaction that will precipitate antigen:antibody complexes in the vicinity of the clone. Clones are then picked with capillaries into 12 well plates as the first step of expansion. Small scale cultures from either type of cloning process are then expanded using culture vessels of increasing volume. Useful amounts of antibody are harvested by maintaining the hybridomas in large volume culture systems, (e.g., large stationary flasks, spinner flasks, hollow fiber reactors), which typically yield 20-100 mg/L, or by injection of the cells into mice for ascites production, which can yield 5-10 mg/ml, but substantially smaller total volumes.

Over the last 20 to 25 years, several innovations have been introduced to the above-described techniques that can be used to make monoclonal antibodies, but these improvements were not combined in a way that would maximally shorten the time from immunization to antibody identification and production. A judicious combination of these innovations is proven to yield IgG-class monoclonal antibodies of high affinity in as little as 5-6 weeks. The specific steps of this FastmAb™ platform include: Simultaneous immunization of each mouse with more than one antigen via injection of pooled recombinant proteins or complex protein mixtures including whole cells or cell fractions of varying complexity, repeated immunization of the mouse at multiple IM sites (RIMMS), or singly into the rear footpad, harvesting of draining lymph nodes rather than, or in addition to, spleen as the source of immune cells, plating of the immune cell: myeloma fusion reaction in bulk into semi-solid medium (methyl cellulose), direct assessment of the presence of clones secreting IgG class antibodies that bind to the antigens during growth of the clones in the methylcellulose, future use of automated harvesting of the antibody-positive clones for scale-up culture. Production of mAbs against TFs will use the FastMab workflow developed. BALB/c mice are used as the source of immune cells, which are fused to a non-secreting myeloma line to generate hybridomas. Hybrid clones secreting IgG were identified, expanded through 96 and 24 well plates, and scaled up to T25 flasks. These flasks yield sufficient volumes of supernatant to assess the properties of the mAbs, and enough cells to freeze down aliquots of each line in liquid nitrogen Immune cells have been harvested from 8 mice and 8 fusions can be performed per week, which yields approximately 2000 total hybridomas. 200 of these hybridomas expand and stabilize, 100 of which are IgG secretors, 40 of which generate ICC signals, and approximately 6 of which are monospecific based on proteome microarray analysis.

### Example 9—Fabrication of Human Antigen Microarrays

To provide sufficient human antigen chips for the proposed project, the −17,000 human antigens as N-terminal GST-His6 fusion proteins from will be purified yeast cells, using an established high-throughput protocol. More specifically the yeast strains will be activated on SC-Ura agar plates and culture in 800 μl of SC-Ura medium overnight in a 96-well format. Each saturated culture will then be inoculated to 12 ml SC-Ura and induced by 2% galactose for 4 hours after the culture reaches O.D. 0.7. The induced yeast cells will be collected and stored at −80° C. To monitor the quality of the induced culture, 24 random strains will be double-inoculated for each batch of culture preparation and run them through the protein purification step first to confirm adequate induction levels. Using immunoblot and silver staining techniques, the success rate of culture induction will be estimated and only those batches that show >85% success rate will be subjected to large-scale purification. Otherwise, culture preparation for failed batches will be repeated. To produce the human antigen chips, the purified human antigen proteins and control proteins, including dilution series of BSA and GST-His6 as negative controls and dilution series of histones, human IgG and IgM, and EBNA1 as positive controls, will be spotted in duplicate on FullMoon slides (FullMoon Biosystems, USA) using two microarrayers, ChipWriter Pro (Bio-Rad) and NanoPrint (TeleChem, USA). The quality of each batch of the chips and the amount of immobilized proteins on the surface will be monitored by probing the chips with anti-GST antibody, followed by Cy3-labeled secondary antibodies. Based on previous experiments, the success rate of printing can consistently reach >90%.

To fabricate the human liver protein chips, these genes were expressed and purified from yeast cells using glutathione affinity chromatography and spotted onto glass slides. Mouse IgG was also spotted on the chips to serve as a landmark. The amount of immobilized human liver proteins on the antigen microarray was visualized and quantified using anti-GST antibodies (FIG. 6). 98.5% (1,042/1,058) of the proteins showed significant signals above the background and that our protein chips are of high quality. To evaluate the specificity of several mAbs generated against different human liver proteins, the chips were blocked with 1% BSA in PBS buffer at room temperature (RT) for a minimum of 1 hr with gentle shaking in a homemade, humidified chamber. Meanwhile, the mAbs purified from ascites were diluted 20,000-fold in phosphate-buffered saline (PBS) buffer. After blocking, the diluted mAbs were added to different chips and incubated at RT for 30 min with gentle shaking. The chips were then washed three times in PBST (1% Tween 20) buffer for 10 min at 42° C. with shaking. Anti-mouse IgG antibodies labeled with Cy3 (Jackson Laboratories, USA, 11,000 dilution in PBS) were added to the chips and incubated in the dark at RT for 1 hr. The chips were then washed 3 times with pre-warmed PBS+0.1°/0 Triton for 10 min each at 42° C. with gentle shaking. After rinsing twice in filter-sterilized, double-deionized water, chips were spun to dryness. To visualize the binding profiles, the chips were scanned with a microarray scanner and further analyzed the binding signals with the GenePix software. As shown in FIG. 3B, the specificity of the mAb generated against pirin was tested against 1,058 proteins spotted on a slide, and this mAb only recognized pirin. Because the amount of each spotted protein does not vary dramatically, this result indicated that the antibody is highly specific. Similar results were also obtained with mAbs generated against four other human proteins.

### Example 10—Validation and Characterization of Monoclonal Antibodies with Identified Antigens

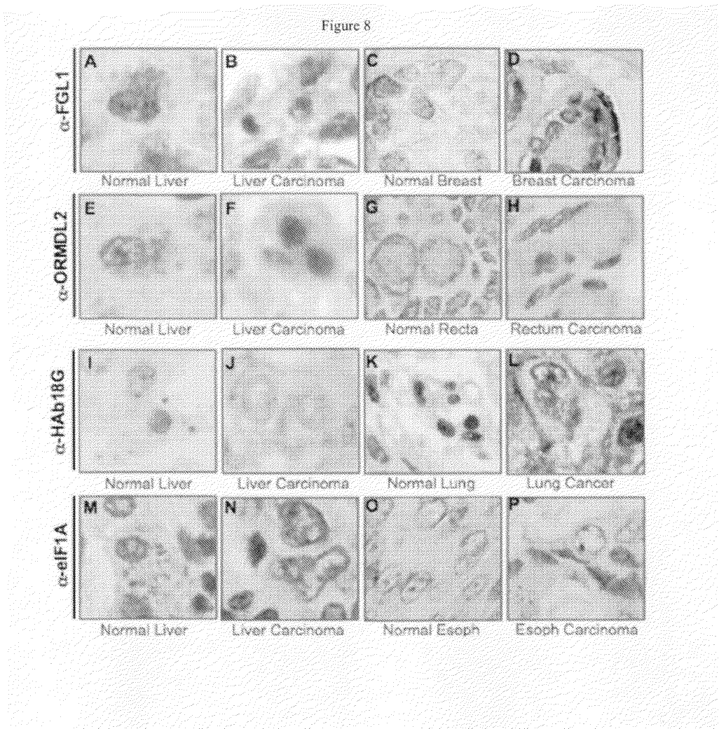

One useful application of mAbs is to profile protein expression patterns using immunohistochemistry (IHC) analysis at various diseased stages (e.g., cancer v. healthy tissue). To demonstrate that the mAbs generated were useful in IHC studies and had potential to be developed into biomarkers, the five mAbs were applied to characterize protein expression patterns of the five identified antigens by using tissue microarrays, each of which contains multiple tumor and normal tissues. All but one (anti-Pirin) showed distinguishable staining patterns in various tissues, and in some tissues they could unambiguously differentiate normal from carcinoma. A close inspection of the results from the IHC staining experiments revealed some interesting expression patterns of the four antigens (FIG. 8). For example, FGL1 can be localized to the Golgi in normal liver; however, in liver carcinoma it is more diffuse and expressed at a lower level (FIG. 8B). ORMDL2 proteins are localized to the ER and excluded from the nucleus in normal liver tissue (FIG. 8E). However, in some liver carcinoma cells the majority of this protein can be nuclear (FIG. 8F). These results suggest that the ER localization of ORMDL2 is aberrant in liver carcinoma, affecting its function in protein folding. Another interesting result comes from HAb18G/CD147, a homolog of the cyclophilin receptor (FIG. 8I-L).

In normal liver, liver carcinoma, and lung cancer tissues, mAb 2B2 showed strong immunostaining (FIG. 8I, J and FIG. 8L), whereas in normal lung tissue, signals were barely detected (FIG. 8K). Staining patterns in normal and liver carcinoma were also different (FIG. 8I). HAb18G/CD147 proteins are almost depleted in the cytoplasm and show dominant localization to plasma membrane in both liver and lung carcinomas (FIG. 8J and FIG. 8L). Interestingly, IHC staining with mAb B4B5C5, recognizing both isoforms of eIF1A (FIG. 8M-P) showed the eIF1A proteins present in almost every cell show granular cytoplasmic staining in normal liver (FIG. 8M) similar to its yeast ortholog. However, it was absent from liver carcinoma (FIG. 8N). Similar differences were observed between normal esophagus and esophagus carcinoma, as eIF1A was undetected in carcinoma (FIG. 8O and FIG. 8P). Unlike its universal staining pattern in normal liver tissues, in normal esophagus tissue, high expression of eIF1A is restricted to polyhedral cells in the middle layer of epithelium, whereas it is not found in the low columnar epithelium or the connective tissues. The above results show how four of five mAbs made by this original method were highly successfully used in IHC, potentially the most challenging application of protein capture reagents, and they were able to reveal a wide variety of interesting differences among distinct type of human cells and tissues.

### Example 11—Validation of Antibodies Against Chromosomal Proteins Using Human Antigen Microarrays

### 1. Determining mAb Specificity

To evaluate the specificity of mAbs generated against human chromosomal proteins, mAbs purified from ascites will be diluted 1000-fold in PBS as a working stock. The chips will first be blocked with 1% BSA in PBS buffer at room temperature (RT) for a minimum of 1 hr with gentle shaking in a homemade, humidified chamber. Properly diluted mAbs in PBS buffer will be incubated on chips at RT for 30 min with gentle shaking. The chips will then be subjected to 3-10 min washes in PBST (1% Tween 20) buffer at 42° C. with shaking. Anti-mouse IgG antibodies labeled with Cy-5 (Jackson Laboratories, USA, 1:1,000 dilution in PBS) will be added to the chips and incubated in the dark at RT for 1 hr. After the same washing step, the chips will be briefly rinsed in filter-sterilized, double-deionized water, and spun to dryness. To visualize the binding profiles, the chips will be scanned with a microarray scanner and further analyze the binding signals with the GenePix software. As a negative control experiment, Cy5-labeled secondary antibodies will be probed to the chips to identify non-specific binding activities. It has been determined that about 150 proteins (e.g., MGMT and PCBP1) showed binding activities to Cy5-labeled secondary antibody against mouse IgG when tested against the 17,000 protein microarrays. The non-specific interactors will be excluded from further analysis.

### 2. Pooling Analysis

Based on preliminary data, a pooling approach will be implemented to rapidly identify mAbs that show monospecificity. To this end, supernatants of hybridomas that secrete high levels of IgG positive mAbs will be combined in “horizontal” and “vertical” pools as shown schematically in FIG. 5. Antibodies can be classified as monospecific if a protein on the microarray is recognized by only a single horizontal and vertical pool that both contain supernatant from the hybridoma in question. This approach has been used to identify 11 out of 88 mAbs tested as truly monospecific, in that they recognize only a single protein on the array. It is anticipated that roughly 10% of all hybridomas screened will indeed show monospecificity. The pooling strategy uses only 2*√x microarrays for microarray analysis, where x is the total number of hybridoma supernatants analyzed. Thus, a total of 14 microarrays can be used to analyze the specificity of 49 different hybridomas, substantially increasing the throughput of the analysis.

### 3. Secondary Screening

Antibodies determined to be monospecific by the analysis of pooled supernatants will then be subject to a round of secondary screening using the protein microarrays, to further characterize both their affinity and specificity. Antibodies that recognize no more than three different proteins on the array will also be set aside for further analysis in this manner, although they may be given lower priority than the monospecific mAbs, as mAbs with highly selective (though not necessarily monospecific) protein recognition properties may also be useful in a range of applications. For these experiments, one human proteome chip will be probed with each mAb at 10,000-fold dilution in the course of secondary screening. If a mAb can specifically recognize its corresponding target protein, meaning no obvious binding signals to any other proteins (excluding those non-24 specific ones as described above) on the chips is observed, it will be probed to a chip again at 50,000-fold dilution to help define an optimal dilution. If no binding signals are observed, the titer will be increased to 100-fold and the chip will be probed again. If binding signals occur, a 1000-fold dilution will be added. Those showing no binding signals at 100-fold dilution will be considered as failures. Therefore, for a typical antigen, 6 chips per antigen (test 3 mAbs per antigen, using 2 chips for each) will be needed for this second round of microarray-based screening.

### Example 12—Generation and Characterization of Monospecific Monoclonal Antibodies (mMAbs) Using a 17,000 Antigen Human Proteome Microarray (Hu-PM)

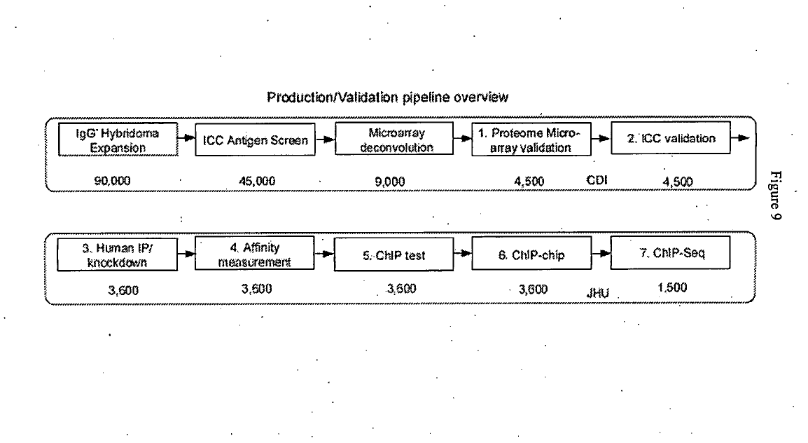

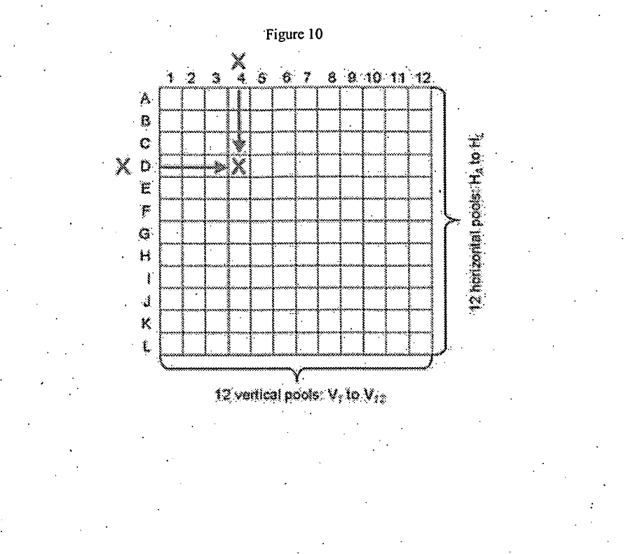

This approach has been commercialized and greatly scaled up and a commercial “pipeline” exploiting this technology is now operational (FIG. 9). The above methods have been substantially improved on, in some respects, by taking advantage of the greatly increased content of the Hu-PMs used and by using a wide variety of innovations, substantially reducing costs of immunization, hybridoma isolation, and screening. The more comprehensive microarray Hu-PM has been used as discussed above with a 17,000 protein content. To aid in alignment and analysis of samples, four spots containing Cy5-coupled human IgG are printed for each block of 750 recombinant proteins. Furthermore, the screening approach has been modified to both avoid the requirement to make ascites or depend on purified antibodies, and to greatly increase overall throughput by using an expanded pooling strategy. In brief, mice have been immunized with both recombinant human proteins and live human cancer cell lines, over 2200 strong IgG-secreting hybridomas have been isolated. To improve the yield of highly specific mAbs, immunocytochemistry (ICC)-based prescreening (FIG. 18) has been conducted for many of these mAbs. This has involved analysis of either the same cell line used for immunization, or in cases where recombinant proteins were used for immunization, cell lines known to express high levels of mRNA for the gene in question. 45% of mAbs have been observed to show a specific ICC signal. This process is referred to as “ICC Screening” in the master pipeline for mMAb validation (FIG. 9). 529 mAbs found to be ICC-positive were then used for Hu-PM-based analysis of specificity, along with 467 mAbs not prescreened in this manner.

### Example 13—Affinity Measurement: Real-Time, Label-Free Detection on a Protein Microarray

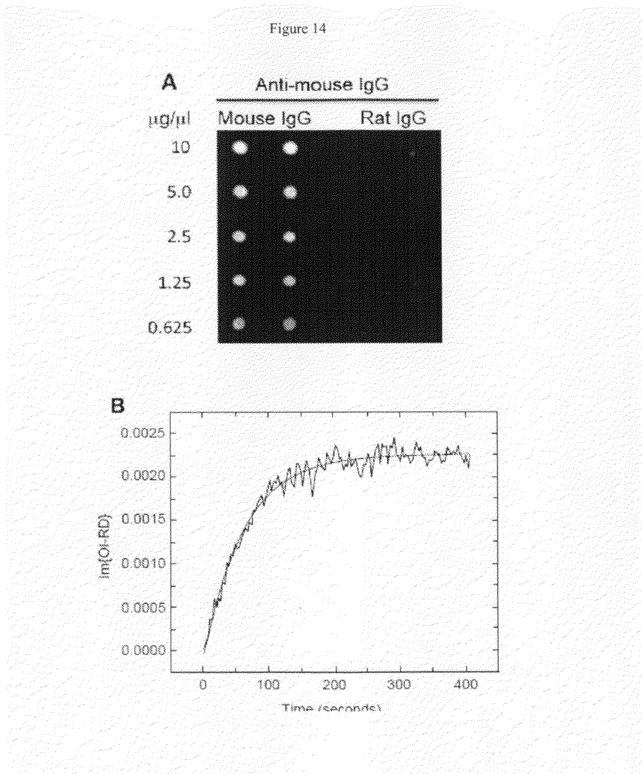

To demonstrate OIRD-based detection of antigen-antibody interactions, a Protein Microarray was fabricated by spotting a simple dilution series of human IgG and bovine BSA, ranging from 0.8 ng/μL to 100 ng/μL, on aldehyde-activated glass. In this configuration the protein microarray was mounted on a translation stage, driven by a computer-controlled high precision stepping motor controlling the x and y directions. To improve sensitivity, the laser beam was first focused on the bare glass surface and the phase shifter was zeroed. Next, the microarray was blocked with 1% glycine in PBS for 1 hr at room temperature, briefly washed with TBST, scanned to obtain a two dimensional (2-D) image^{Blocked} of the microarray. After 1 hr of incubation with goat anti-mouse IgG at 20 μg/mL, the microarray was subjected to PBST and PBS washes and scanned to obtain a two-dimensional image^{Binding}. The final binding signals were then determined by subtracting image^{Blocked} from image^{Binding}. FIG. 14A is a computer-regenerated differential image. Further analysis of the OIRD signals shows that the detection limit was at least 20 fg on a 120 micron spot with very high reproducibility. In addition, the averaged intensity of each spot is in a linear relationship with the amount of antibodies spotted. More importantly, rather than rastering across the entire glass slide, which would be extremely time consuming, the laser beam can be assigned to a particular subregion of the slide for detection of real-time binding intensity for an individual protein spot (FIG. 14B). Therefore, this method is proven useful for directly determining affinity/avidity values of antibodies in a protein microarray format. These observations form the basis of the Affinity Validation Step of the pipeline (FIG. 9).

### Example 14—ChIP

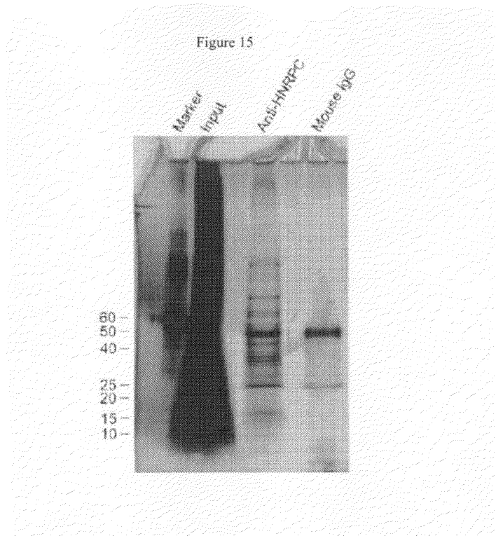

A proof of principle experiment was performed to see whether an anti-TF antibody's ability to perform ChIP can be generically tested. Anti-HNRPC, an “unconventional” transcription factor, is used and pull-down of many specific bands on a silver stain gel is observed, including histone-size bands (FIG. 15). Consistent with this possibility, following deproteinization, a simple measurement of DNA content using a Nanodrop shows a five-fold higher amount in the HNRPC sample, suggesting this simple test can be adapted to high throughput.

### Example 15—Antigen Acquisition: The Case for Protein Domains as Antigens

It is possible to produce very large amounts of small protein domains, which can fold into native structures and have been highly successful for structure determination and sufficient protein can be produced to satisfy the needs of the current disclosure. In some instances antibodies raised against human transcription factor (TF) domains may lead to a lower yield of antibodies that recognize the native protein. Mouse and human proteins can be rather similar. Since self-surfaces are not generally good antigens, this may tend to direct the immune response toward the “non-natural” surfaces of the protein domain, namely those hidden in the completely folded protein, and the terminal segments, which are likely to be peptide-like. Peptides make very good antigens for IB but may not work as well for IP or ICC. The outlined approach is supplemented with a combination of full-length recombinant proteins and chromatin purified from a variety of cell types. Instead of using large quantity of purified antigens to generate protein affinity reagents, a “chromatin shotgun” approach was utilized, in which crude chromatin is used to immunize animals directly. An advantage of this approach is to obviate the need for producing large amount of purified antigen for immunization. Chromatin from approximately 100 diverse human cell lines is available, including the 60 NCI cancer cell lines, and can be used as immunogens. The rationale for choosing crude chromatin immunogens is several-fold. Antibodies are widely used in diagnostic applications for clinical medicine (e.g., ELISA and radioimmunoassay systems). Analysis of cells and tissues in pathology laboratories includes use of antibodies on tissue sections and flow cytometry analyses. mAbs can be expected to preferentially recognize tumors or cancer cells in ICC and/or IHC assays and may be more likely to be developed as biomarkers for cancer diagnosis and/or prognosis. Furthermore, if the chromatin is prepared just as for ChIP analysis, it may lead to a much higher likelihood of ChIP-grade mAbs because the TF DNA binding surfaces can be occluded. Several obvious advantages follow: There is no need to clone or purify any protein in high quantity; proteins are untagged and therefore, in a native form and conformation, many distinct types of cells can be used; and the chromatin shotgun approach is rather efficient allowing rapid generation of a large number of hybridomas that can recognize many antigens. The chromatin shotgun approach can be practical when a feasible deconvolution method is available, (e.g. the use of the Hu-PMs). An expected improvement is to better evaluate behavior of the pipeline at immunization. Mice with five distinct human cell lines (as opposed to chromatin) have been immunized, resulting in identification of 91 mMAbs against 82 unique and diverse antigens. Different cellular backgrounds may affect outcomes of mAb generation because chromatin protein profiles of different cell lines or tissues can vary dramatically, and presumably will improve comprehensiveness of the mAbs produced. On the basis of results obtained via use of chromatin immunogens, evaluation of whether reducing immunogen complexity would further boost the immune response, and thus help produce more, diverse mAbs is possible.

### Example 16—Immunizations

As outlined in the previous sections, two or more different forms of protein immunogens can be used to generate TF-specific mAbs. In one formulation, purified recombinant forms of whole TFs or TF protein domains (some of which can be affinity-tagged), can be combined into pools of 3-6 proteins containing equivalent amounts of each protein, mixed with Titermax adjuvant, and injected either subcutaneously in a plurality of dorsal and/or ventral sites, followed by boosting two and four weeks later and harvesting of the spleen for immune cells, or into a rear footpad. This can be followed either by harvesting of popliteal lymph node (PLN) 14-16 days later, or boosting and harvesting of PLN for immune cells two weeks after that. The PLN approach is efficient at generating IgG-secreting immune cells against antigens in complex mixtures of proteins as are found in whole cell lysates, and can also generate IgG secreting cells in response to small pools of defined recombinant proteins. In the second formulation, immunogens can comprise preparations of chromatin isolated from fixed nuclei. Whole cell extract immunogens can yield anti-TF mAbs in the expected approx. 8% yield of total anti-cell protein mAbs, consistent with the percentage of TF genes in the human proteome. Use of nuclei or chromatin as immunogens can greatly increase the yield of TF-specific Mabs over total cell immunization.

### Example 17—Harvest and Fusion

Immune cells from either spleen or PLN can be collected as a suspension of cells found in each organ. This mixture can be fused to a non-secreting myeloma cell line via PEG. This standard fusion method can be sufficient to yield >2000 hybridomas per fusion, a substantial but small fraction of which are IgG-secretors. Two process improvements can be standardized to enhance hybridoma production efficiency. Carbohydrate beads containing ferritic particles (MACS from Miltenyi Biotec, or Dynabeads from Invitrogen) will be used to enrich for IgG-secreting plasma cells found in the immune spleen and PLN cell suspensions. This will be done using a two step method that first removes non-plasma cells via biotinylated antibodies specific for T cell markers plus avidin-coupled magnetic beads, and in the second, directly enriches for plasma cells via another set of magnetic beads coupled with anti-CD138 (syndecan) antibody. This method is capable of enriching plasma cells 100-10,000 fold out of a mixed spleen cell lysate. Test runs can yield approximately 1000 fold enrichment of plasma cells from both spleen and PLN lysates. This enriched cell population will then be fused to myeloma cells to generate hybridomas. In the second improvement, immune cells will be fused to myeloma cells using an electrofusion apparatus (Eppendorf; NEPA GENE, LF201 Electro Cell Fusion Generator; and BTX, ECM 2001 Electro Cell Manipulator) instead of PEG. In this method, the cells to be fused can be combined in a hypotonic solution, aligned into “chains” within a strong electric field in the electrode chamber, and shocked with an electric pulse to meld the membranes. This method allows for the creation of hybrids starting with far fewer immune and myeloma cells. The combination of enrichment for plasma cells plus electroporation-mediated fusion may increase the percentage of the hybridomas that secrete antibody, and reduce the number of irrelevant clones that need to be carried in the downstream 96 and 24 well expansion steps

### Example 18—Plating and Identification of Clones

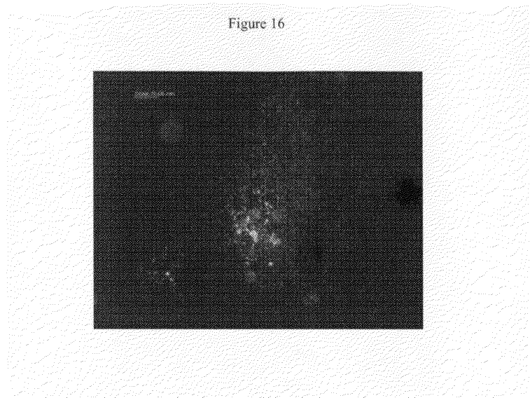

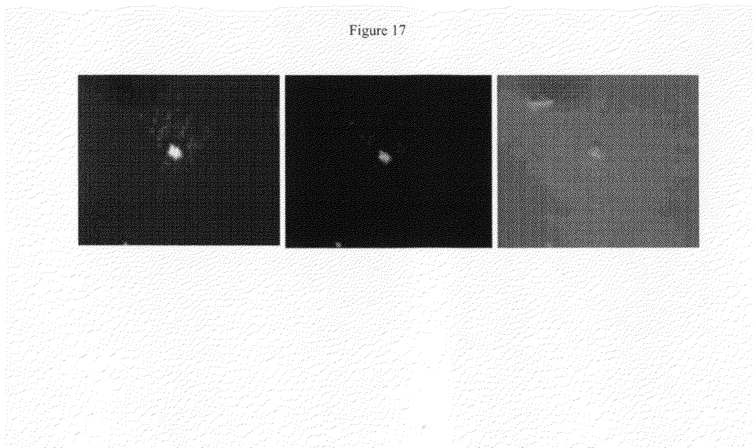

Cells contained in the fusion reactions were plated in medium that selects for outgrowth of hybrids comprising an HPRT(+) immune cell from the mouse and an HPRT(−) myeloma cell. Fusion reactions were directly plated into this selective medium to which is added methylcellulose (MC), resulting in a semi-solid medium supporting growth of physically separated clones of hybridomas. These clones were isolated directly from the MC using microcapillary tubes and plated into 96 well plates for expansion. The use of MC to grow the hybridomas circumvents the need for establishing clonality by the time-consuming limiting dilution method, which would add 2-3 weeks to the process. Fluorescently tagged anti-mouse IgG were utilized in the MC selection medium, (FIG. 16), permitting direct identification of antibody (and IgG vs. IgM) secreting clones. This can reduce the number of clones to be handled in 96 and 24 well plates. Two further modifications to this methodology were incorporated into the MC growth step. Anti-GST antibodies can account for up to 30% of the IgG secreting clones when GST fusion proteins are used as immunogens. Recombinant GST protein tagged with DyLight 549 (Pierce) were added to the selective MC medium to tag those colonies secreting antibodies against epitopes found on the GST portion of the recombinant TF fusion proteins. This can eliminate the need to carry these irrelevant clones through subsequent culture expansion steps. A GST-tolerant line of BALB/c mice will be developed to eliminate the immune response of the immunized animals to the GST portion of the recombinant TF fusion proteins. In the second modification, the proteins in the pool of antigens used to immunize the donor of the spleen or PLN for that fusion can themselves be coupled with DyLight 488 and added to the MC to tag colonies secreting antibody against the fusion proteins. The plates containing colonies growing in MC can be viewed with an inverted fluorescence dissecting microscope, and the GST-549(−)/TF-488(+) colonies picked for expansion (FIG. 17). In this reconstruction experiment, although anti-GST antibodies were detected, specific antigens or antigen blends can be coupled to the DyLight-549, in which case double-positive cells can be selected, which can lead to a substantial impact on the yield of mMAbs against recombinant antigens. Harvesting of fluorescently tagged colonies can be done using an automated microscope-camera-robotic picker system, such as the CellCelector made by Aviso, which can extract colonies from MC. Labeled colonies can be distinguished and 100-200 colonies can be hand-harvested per hour.

### Example 19—Clonal Expansion

The expansion of clones from 96 well plates to 24 well plates can take approximately two weeks. At the end of this scale of growth, a sufficient volume of antibody-containing supernatant is available to assess the binding specificity of the mAbs. Expansion of the clones of interest continues by transfer to T25 flasks, from which cells are frozen for permanent storage and supernatant is collected for further analyses. Expansion to the T150 flasks yields antibody containing supernatant (20-100 μg/ml) on a sufficient scale (5 flasks yield 1 liter of supernatant, or 10-50 mg IgG) to affinity purify with protein G-resin and store as inventory. Larger scale inventory can be rapidly generated using spinner flasks to create culture supernatant with high concentrations of antibody, followed by affinity resin purification.

### Example 20—Human Proteome Microarray Validation for Monospecificity

The Hu-PM generated can be used to quantitatively assess the specificity of mAbs generated. Supernatants from IgG-secreting hybridomas that show strong binding to target TFs by ELISA will be harvested and frozen. A minimum of three mAbs can be tested for each TF, requiring a minimum of 9000 hybridoma supernatants to be screened, as only about half represent the desired monospecific mAbs (mMAbs). Batches comprising 144 different supernatants will then be tested, in which individual supernatants can be combined in 12×12 two-dimensional pools. For each batch of 144 supernatants, proteins bound by each pool can be identified as follows. The Hu-PMs will be blocked with 1% BSA for 1 hr with gentle shaking in a custom made humidified chamber. After blocking, the “row” and “column” pools of mAbs will be added to different Hu-PMs and incubated with gentle shaking. The Hu-PMs will be washed and anti-mouse IgG antibodies labeled with Cy3 (Jackson Laboratories, USA, 1:1,000 dilution in PBS) will be added and incubated in the dark at RT for 1 h. The Hu-PMs will then be washed and rinsed. After rinsing twice in water, Hu-PMs will be spun to dryness. To visualize binding profiles, Hu-PMs will be scanned and binding signals will be analyzed. To quantitatively evaluate intensity and specificity of mAb binding, the 38 proteins that showed non-specific binding to all IgGs in previous assays will be visually flagged. The mean foreground signal intensity across the Hu-PM will then be calculated; excluding nonspecific hits, empty wells, and positive control spots (e.g. mouse IgG, Cy5 dye, etc) from analysis, and the number of standard deviations above or below the mean for each spot on the array will be determined. All proteins for which both replicate spots show signal intensity a minimum of five standard deviations above the mean will then be scored as positive. Deconvolution of this analysis of pooled samples will be performed to assess antibody specificity. This analysis will be used to determine which mAbs against a given TF show high specificity, i.e. qualify as mMAbs. These supernatants will then be tested individually on the array. The binding intensity and specificity of individual mAbs will be determined by evaluating S, the number of standard deviations (SD) above the mean for the top hit on the array, and the difference in the number of SDs between the top hit on the array and the second-best hit. Several improvements can improve throughput for this step in the pipeline. The yield of purified recombinant protein used in microarray fabrication will be improved by using multiple rounds of elution from the glutathione resin used for affinity purification. The yield can be at least doubled in this manner. The amount of protein wasted in the printing process can be reduce by increasing the number of slides printed in one run from each batch of eluted protein, thus reducing evaporation. Protein preparations can be stored at −80° C. in humidified dessicator boxes to minimize loss of product from sublimation. These modifications may lead to at least a two-fold reduction in costs of protein production, the most costly component of Hu-PM production. Other improvements can include expanding pool size from 12×12 to 24×24 in a two-dimensional pooling design, or using three-dimensional pools. The overall sensitivity of the screen can be improved by concentrating the supernatants five-fold prior to pooling by use of sample concentration columns. This can lead to improvement of throughput by at least two-fold for deconvolution probing.

### Example 21—Characterize mMAbs by ICC Validation and Database Images

One screening step for useful antibodies is an immunocytochemistry (ICC) assessment of the ability of the antibodies to bind proteins in fixed cells. Hybridoma culture supernatants from clones expanded through the 24 well to T25 flask stages contain sufficient antibody (tens of μg/mL) to carry out screening of fixed tissue culture cells for in situ binding to target antigens. This ICC screening was done by adhering various cell lines (ATCC no. CCL-2, CCL-247, HB 8065, HTB 22, CCL-240) to 16-chamber glass slides coated with polylysine, fixing with 4% paraformaldehyde, blocking, and incubating with neat culture supernatant or concentrated antibody diluted to approx. 10 μg/mL in blocking solution. Binding of this primary antibody was detected with DyLight488-coupled anti-mouse IgG, and then viewed and photographed using a fluorescence microscope-camera system (FIG. 18). The positive control reaction was an anti-β actin mAb primary antibody, and a negative control for background was a well to which no primary antibody has been added. Images representing the typical binding pattern of a given mAb were recorded in a database as .gif files with file names that include the antibody clone identifier. Binding image descriptions were characterized and recorded with respect to the intracellular binding site or pattern (e.g. nuclear, plasma membrane, perinuclear, cytoplasmic diffuse, filamentous or punctuate, etc.). Multiple locations of binding within subpopulations of the plated cells, which may indicate cell cycle-specific relocation, were also noted. ICC can be used as one of the earliest screening steps to identify mAbs that react with native antigen. This screening step uses the above-mentioned range of cell types as targets to identify mAbs with native antigen binding ability before assessing antigen specificity on the Hu-PM, decreasing the number of Hu-PMs needed. An improvement on the overall production process at the ICC stage will be to take advantage of the subset of the Human Proteome Expression Library cultured cells that contain plasmids encoding the individual TF genes. Those cell lines expressing the TFs that were in a given antigen pool will be plated, as a pool of cells and screened by ICC to identify those mAbs that are capable of binding to a TF in the pool. As the cells used in the ICC screens can be aldehyde-fixed, this screening method can directly identify mAbs useful in ChIP techniques using formaldehyde fixed chromatin. If TFs are expressed at too high a level in the cell lines, there is the possibility that not all the TF proteins in the cell will be normally associated with DNA and other factors, and might thereby expose epitopes normally hidden. The amounts of the overexpressed TFs can be controlled with the concentration of Tet to lower expression levels if there is substantial staining of the mAb outside the nucleus. Even with this caveat, use of these clones as a screening tool represents a powerful tool to quickly identify those mAbs that have any binding activity toward the TFs in a particular antigen pool.

### Example 22—Mass Spectrometry (MS) Validation of Protein Identification on Purified Recombinant Protein Targets

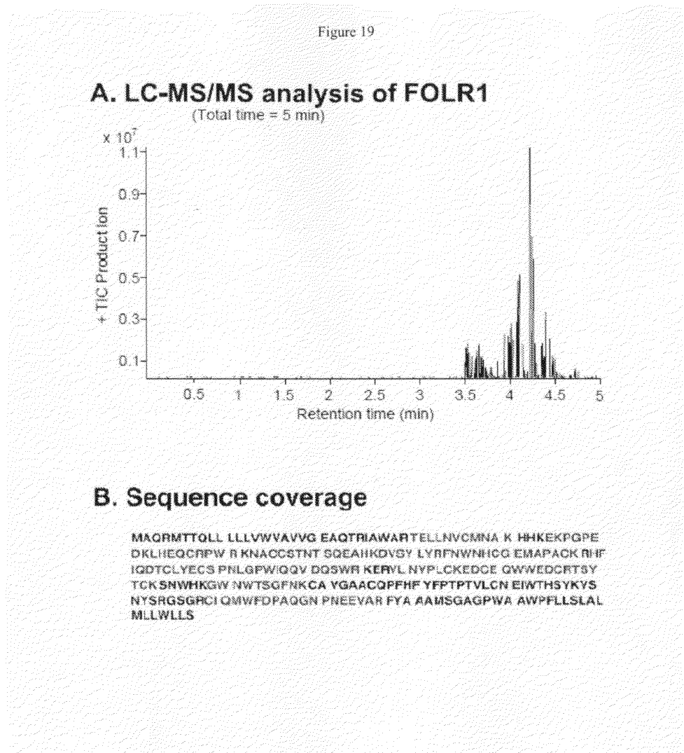

A final validation of the protein identification provided by the Hu-PM will be to perform MS on the protein preparations that are used to make Hu-PMs. This will be done for all 1500 TFs. MS-based “shotgun proteomics” will be employed, in which proteins are proteolytically cleaved by trypsin into peptides followed by sequencing of peptides by tandem MS interfaced with liquid chromatography (LC-MS/MS). In such “bottom-up proteomics”, the fragmentation technique can be collision induced dissociation (CID) in which the collision of peptides with inert gas molecules results in dissociation of the peptide backbone amide bonds, between the carbonyl and the amine groups. Assignment of individual MS/MS spectra to individual peptide sequences will be done using spectrum-to-sequence database search algorithms. A high resolution quadrupole time-of-flight (QTOF) MS can be an instrument platform to carry out CID-based tandem MS analysis of the purified proteins. The MS will be fitted with a chip cube, which facilitates rapid and comprehensive MS/MS analysis of fmol amounts of proteins (FIG. 19). A possible drawback of identifying peptides using tandem spectra will be statistical introduction of false positives (e.g. 1% FDR) because lack of spectral references can lead to database search using algorithms instead of spectral matching. Thus, constructing a spectral library can be helpful for further proteomic research as well as systematic validation of candidate biomarkers. In these experiments, not only spectral data representing the TFs will be acquired, a mass spectral library using annotated spectra as a reference will be constructed. The spectral data will be processed by proper analysis platforms and the peptide sequence, charge state, protein name, sequence accession number, score/e-value and name of the search algorithms used will be annotated.

### Example 23—Validation and OC: Immunoblot/IP in Human Cells

Each monospecific mAb (mMAb) will be tested for its ability to perform immunoprecipitation (IP) (FIG. 12 and FIG. 13). A medium throughput mMAb validation pipeline for IP has been developed, which exploits the 18,500 proteins produced in yeast. For each mMAb, a 50 mL culture of the same yeast strain expressing the antigen of interest as an N-terminal GST fusion (i.e. the antigen identified in the microarray validation step) will be grown up, and a crude protein lysate will be prepared by using a Microfluidizer in extraction buffer. The lysate will be IP'd with the mMAb and the IP will be subjected to SDS PAGE and immunoblotting with an anti-GST antibody. To improve, streamline, and reduce costs in evaluating IP ability, a variety of different methods of preparing the yeast lysate, will be evaluated to see whether the starting culture will be downsized to 10 mL. The induction conditions, experimenting with various pre-growth regimens such as 0.1% glucose, 0.2% glucose and 1% raffinose, as well as the dilution factor when the overnight culture is induced will be varied and tested. The cell breakage in a mini bead-beater, which will be far more efficient than the method currently in use, can also be performed.

### Example 24—Affinity Validation

A commercial source, for example Affina, will be utilized for measurement of the antibody affinities. Affina uses both Bio-Layer Interferometry (BLI) and Surface Plasmon Resonance (SPR) methods to make these measurements. BLI is an optical technique for measuring molecular interactions. The BLI instrument analyzes the interference pattern of white light reflected from two surfaces: a layer of immobilized protein on the biosensor tip, and an internal reference layer. A change in the number of molecules bound to the biosensor tip causes a shift in the interference pattern that will be measured. Binding between a ligand immobilized on the biosensor tip surface and an analyte in solution produces an increase in optical thickness at the biosensor tip, resulting in a wavelength shift which is a direct measure of the change in thickness of the biological layer. ForteBio BLI instruments contain eight light conducting probes which will be monitored simultaneously, thus measuring eight association and dissociation rates. Interactions are measured in real time, providing the ability to monitor binding specificity, rates of association and dissociation, or concentration. For the current study an antibody to be tested will be captured on anti-human or anti-mouse antibody capture tips, transferred into wells containing a range of antigen dilutions to measure the association rate and then to wells containing buffer to measure the rate of dissociation. Each antibody will be tested at multiple concentrations of antigen and one buffer reference. As an alternative method, SPR instrumentation will be used for analyzing antigen/antibody interactions with a FastStep pioneered by ICx Technologies.

### Example 25—ChIP-Chip and ChIP-Seq Validation

A two-tiered procedure for advanced ChIP analysis has been designed, starting with a ChIP-chip on a promoter array and a more comprehensive ChIPSeq approach. These procedures will be done on the human cell line expressing the TF in question.

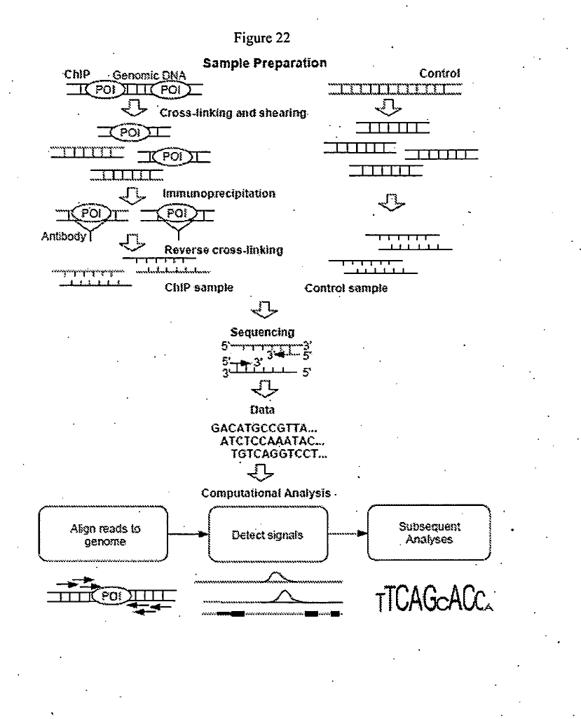

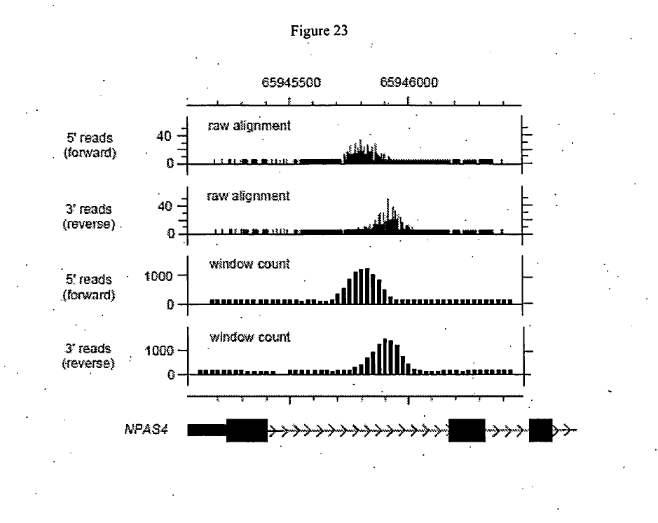

1. ChIP-chip: As the second tier of ChIP analysis, a custom promoter miniarray will be designed, allowing profiling of 8 samples/slide. Arrays will be designed using eARRAY and will tile about 25% of human promoters, including ENCODE regions and randomly chosen genes to make up the remainder of the array real estate. A “fixed” ChIP-chip protocol will be performed that requires the TF, e.g. TF#1, to be formaldehyde cross-linked to chromatin from the HeLa cell line expressing TF#1. Chromatin will then be sheared into ˜500 bp pieces (Covaris) followed by IP of TF#1 bound chromatin fragments using a TF#1 specific mAb. Next, crosslinking between TF#1 and bound chromatin will be reversed, resulting in the ChIP sample of interest. Negative control HeLa samples will be made in parallel, one following the same protocol but eliminating the primary antibody and a second, total DNA sample (FIG. 22). A comparison of control and ChIP samples should show an enrichment of DNA fragments bound to TF1 in the ChIP samples, providing an estimate of noise in the assay.
2. ChIP-Seq: ChIP-sequencing or ChIP-seq can be a method to analyze protein-DNA interactions using chromatin immunoprecipitation followed by massively parallel high throughput sequencing. The 1500 TFs with “cistrome” or cis-acting DNA elements will be identified using the Illumina HiSeq. The ChIP-seq sample prep and experimental design can be similar to that described for ChIP-chip. The ends of DNA fragments from both control and ChIP samples will be sequenced using Illumina's HiSeq resulting in millions of 100 by reads. These reads can be uniquely and efficiently mapped to the human genome using open source software, such as Bowtie (http://bowtie.cbcb.umd.edu). Once mapped, genomic regions in which the ChIP reads are statistically enriched will be identified. CisGenome (http://www.biostat.jhsph.edu/˜hji/cisgenome), another open source tool can provide a user-friendly interface to convert alignment file formats, model background noise, i.e. ChIP sample read enriched peaks and to functionally annotate and visualize genomic regions of interest. CisGenome uses a binomial model as its background noise model for two (ChIP and control) sample experiments. Using CisGenome, the reference genome will be scanned into 100 bp long non-overlapping windows with a local false discovery rate (FDR) and fold enrichment (ChIP to control read counts) computed for each window. All windows with a local FDR smaller than a given cutoff (<=10%) will then be selected. Overlapping windows, which can be defined by the user, will be merged into a single region. The biggest fold enrichment among the overlapped windows will be assigned as the fold change of the merged region. All final merged regions will be reported as the output. Legitimate transcription factor binding sites (TFBS) will have a characteristic bimodal peak shape, ie, with a peak just upstream and downstream of the binding site (FIG. 23). In order to determine the binding site boundary, CisGenome can scan a given output region with a user-defined sliding window size counting ChIP sample reads aligning in the forward and reverse orientations and then creating two smooth curves of read counts for each (forward and reverse) respectively. The modes of the curves will then used to determine the boundaries of the binding sites. Based on the binding site boundaries, CisGenome can determine other statistics like median DNA fragment size.

### Example 26

We will to evaluate the presence of biomarkers using a novel protein chip technology to characterize proteomic profiles from normal and diseased tissue, and to rapidly develop monoclonal antibodies (mAbs) against the candidate biomarkers with a rapid approach that that is currently being used at CDI. Using this exceptional platform, discovery-based screening of biological samples for novel and known antigens can be conducted in a high-throughput fashion, allowing rapid and specific detection of mAbs raised differentially in diseased versus control tissues.

The main objective of this project is to identify TF-specific protein biomarkers and to rapidly generate affinity reagents, in this case mAbs, against the biomarkers using samples with and without a disease, in order to characterize the disease-specific antibodies generated and expressed in mice sera that detect novel antigens within the tissues of diseased patients. Specifically, through this project we will elicit an immune response in mice to samples from patients with and without a disease, in order to generate disease-specific antibodies and therefore an transcription factor proteomic signature in the sera of the immunized mice. The immune response of the mice will be decoded with a human proteome microarray. Additionally, mAbs against the novel or known disease-specific proteins will be generated that can be used in further studies.

We propose to take advantage of the recent development of a protein chip containing 17,500 yeast-derived recombinant human proteins to screen previously immunized mouse sera for anti-endometriotic tissue mAbs in a high-throughput, highly-specific manner. This proprietary platform from CDI will allow the identification of a signature of proteins expressed by disease tissues but not by normal tissues that would represent possible diagnostic and therapeutic targets for the disease. Additionally, the platform provides for the generation of mAbs against the identified biomarkers for immediate validation for their usefulness in disease characterization using standard, widely-used techniques including immunohistochemistry (IHC), Western blot (WB), chromatin immunoprecipitation (ChIP) and immunoprecipitation (IP), among many.

A panel of TF-specific mAbs will be generated upon immunization of mice with diseased tissues, which will represent a protein signature for the disease. Identification of TF-specific protein signature will help advance the field by discovering potential diagnostic and therapeutic targets for the disease.

The transcriptome that characterizes a particular disease is only part of the story; it is now widely accepted that global gene expression studies must be validated by protein studies in order to take into account post-transcriptional regulation changes that may not be detected by a cDNA microarray platform. Protein studies can accurately show which gene expression changes result in measurable changes in protein levels that ultimately may be causative of disease. Because of the high specificity and accuracy of the proteomic approach proposed herein, we strongly believe it has a great potential to gain novel insights into the transcription factors underlying diseases by identifying proteins that are over- or underexpressed in diseased samples in a relatively rapid and highly specific manner.

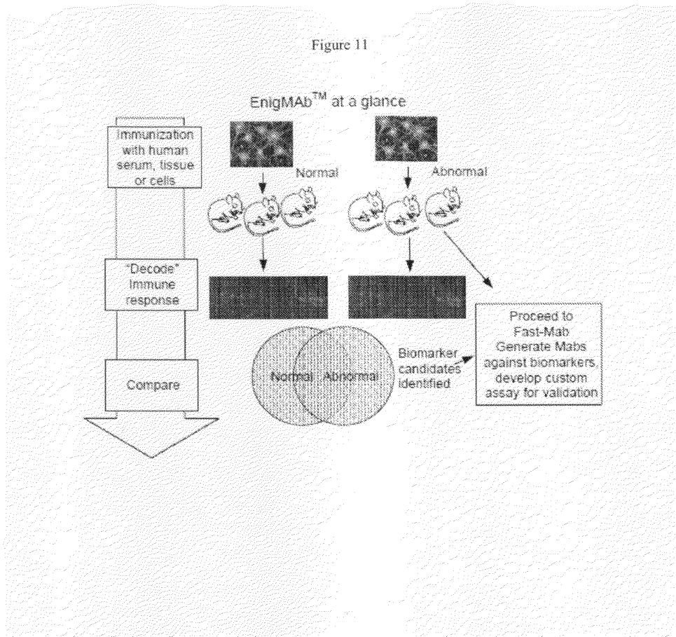

This study entails the development and identification of transcription factor biomarkers by way of profiling mice immune responses to samples from normal and abnormal tissue mAbs using a new, innovative and unique protein chip technology available at CDI (FIG. 11). Various approaches have been used to investigate protein expression profiles in diseases, including endometriosis, which consist of the use of 2D gel electrophoresis followed by protein identification using mass spectrometry (MS), matrix-assisted laser desorption/ionization time-of-flight mass spectrometry (MALDI-TOF-MS) combined with magnetic beads, and biochips. These approaches are limited in various ways. For some, protein identity has to be undertaken by additional steps such as on-chip peptide sequencing. Others have limited resolution of low-molecular weight proteins. Approaches that use chips with different surface chemistries require many steps and several chip types to completely characterize a sample's proteome. As a result, the great potential of proteomic technologies for the elucidation of the mechanisms at play in endometriosis has not reached its maximum. The proposed studies are innovative in that they will apply a new, unique and accurate protein profiling technology to identify novel biomarkers coupled to high-throughput monoclonal antibody generating platform, currently used to generate highly specific antibodies that have either no cross-reactivity with other human proteins or that its cross-reactivity profile is known, as it is also profiled against the proteome-wide chip, thus resulting in a better capture reagent with better performance in downstream immune-based applications (IHC, WB, ChIP and IP). Also, because this technology is high throughput, it simplifies and expedites in a cost-effective manner the discovery of a protein signature of a diseased tissue while at the same time generating specific Abs that can be used in downstream applications and investigations on the significance of the aberrant levels of the proteins discovered.

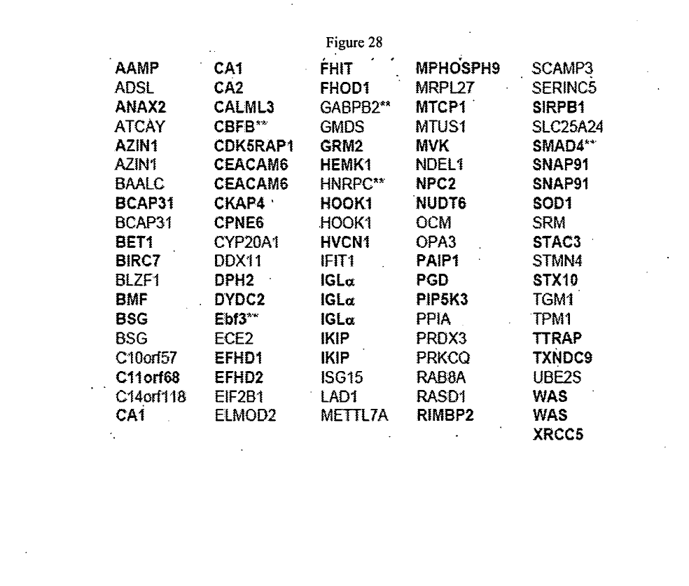

To generate mAbs in mice in a high-throughput fashion, CDI has developed a “shotgun” immunization method coupled with protein chip technology to deconvolute the corresponding antigens. Using this approach, live cells from human cancer cell lines have been used as immunogens to immunize mice. After periods that range from 14 to 45 days, lymphoid cells from mice are isolated and fused with myeloma cells to generate hybridomas (500-1,000 per fusion). Human cell lines are then used to screen the hybridomas for binding activity by way of immunofluorescence. From the initial screening, about 40% of mAbs are found to recognize cellular antigens. To identify their corresponding antigens, the hybridomas that recognize human native antigens in the cell screen are then tested against a human protein chip which contains 17,500 yeast-derived recombinant human proteins. To rapidly identify the corresponding antigens a proprietary pooling technique is utilized to screen the supernatants against the protein chip. In this fashion the company has generated more than 100 mAbs (Table 1) including six against sequence-specific DNA binding proteins (FIG. 28). Furthermore, the specificity of each mAb identified is then reconfirmed against the protein chip and only monospecific CDI has immuned not only with human cells, but with serum and serum fractions. From additional work done immunizing with pooled recombinant human proteins in which mAbs have been successfully generated, it is expected that the use of serum as immunogen will elicit an immune response in animals.

### Example 31

Recently a variant of its approach has been developed in which the proteome chip is utilized to identify, at the mouse serum level, the antibodies that have been generated against a particular antigen or group of antigens, prior to the hybridoma generation stage. By comparing results from protein chip analysis of animals immunized with normal or diseased tissue it is possible to identify subtle differences in the antibodies generated by each individual. These differences are likely to be specific to the particularities of the sample which expresses them.

Diseased and non diseased biological samples will be injected into mice (one tissue per mouse) with the objective of eliciting an immune response. Abs generated will be identified at the serum level with high accuracy by probing a protein chip containing 17,500 yeast-derived human proteins in duplicate. Differentially-expressed proteins detected will be rapidly expanded form CDI's proteome-wide the yeast expression library and the mice previously immunized and that have shown the strongest differential expression among the experimental groups, will be boosted with diseased tissue will be boosted with selected recombinant protein(s) showing the strongest differential expression among the experimental groups. Hybridomas will be generated and their specificity against the selected proteins tested and further validated for their applicability further in techniques including IHC, WB, IP, and ChIP. Approximately 6-10 BALB/c mice (3-5 per experimental group) will be used for the studies.

Immunization scheme: Using the novel platform created by CDI, a strategy will be undertaken to determine the feasibility of generating mAbs that detect disease-specific antigens. Starting at 2 weeks after immunization of mice with tissues, serum from the immunized mice from the two experimental groups (disease vs. control) will be obtained, aliquoted and stored for further analyses. At appropriate time intervals (e.g., every two weeks, after boost immunizations if required as determined by WB), WB analyses using standard methods will be performed to monitor the generation of appropriate amounts of Abs.

Fractionation and profiling of antiserum: Next, frozen aliquots of serum collected from mice. Samples will be processed so as to remove common proteins present in serum (e.g., albumin; IgG) using a HAS/IgG affinity resin, and pooled according to experimental group. A key step will be to profile the serum samples against the whole human proteome microarray. This will allow for identification of potential endometriosis biomarkers and also identify antigens for additional boost immunizations. Specifically, Abs generated will be identified at the serum level with high accuracy by probing a protein chip containing 17,500 yeast-derived human proteins in duplicate. To evaluate the antibody profile generated against the different samples, the serum samples will be diluted 1000-fold in PBS as a working stock. We will first block the chips with 1% BSA in PBS buffer at room temperature (RT) for a minimum of 1 hr with gentle shaking in a homemade, humidified chamber. Properly diluted serum samples in PBS buffer will be incubated on chips at RT for 30 min with gentle shaking. The chips will then be subjected to 3-10 min washes in PBST (1% Tween 20) buffer at 42° C. with shaking. Anti-mouse IgG antibodies labeled with Cy5 (Jackson Laboratories, USA, 1:1,000 dilution in PBS) will be added to the chips and incubated in the dark at RT for 1 hr. After the same washing step, the chips will be briefly rinsed in filter-sterilized, double-deionized water, and spun to dryness. To visualize the binding profiles, we will scan the chips with a microarray scanner and further analyze the binding signals with the GenePix software. As a negative control experiment, Cy5-labeled secondary antibodies will be probed to the chips to identify non-specific binding activities. Differentially-expressed proteins detected will be rapidly expanded from the yeast expression library and the mice previously immunized with disease tissue will be boosted with a panel of selected recombinant protein(s) showing the strongest differential expression among the experimental groups. Boost immunizations will involve utilizing recombinant proteins already available at CDI.

Hybridoma generation: Subsequent steps will involve the generation of hybridomas for the production of disease-specific mAbs. Hybridomas will be generated using proprietary methods previously described. Briefly, antibody-secreting hybridomas will be generated by fusion of B-cells with myeloma cells. The identity and specificity of generated monoclonal antibodies will be conducted by profiling with the protein microarray as previously described.

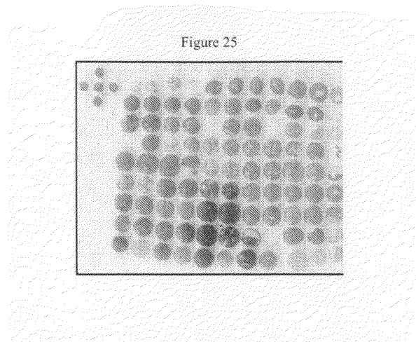

Validation of biomarkers: Validation and verification steps will include carrying out WB or other appropriate antibody-based detection methods on a larger cohort of patient samples. We propose to test the specificity of the antibodies generated with this approach using WB of protein extracted from additional disease and experimental tissues (up to 10 samples per group). In addition, disease-specific mAbs will be further validated by IHC (FIG. 26 and FIG. 27) using a disease-specific tissue microarray (TMA) (FIG. 25) This will allow the validation of the specificity of the mAbs for the disease and will also reveal possible differences in the expression of biomarkers by lesions of varying localization.

Data analysis: Data mining and bioinformatic analysis of data will be obtained in order to generate an disease-specific signature

1. A method of identifying one or more biomarkers comprising:
(a) administering to a first non-human animal a first biological sample;
(b) comparing an immune response from the first non-human animal to an immune response from a second non-human animal administered a second biological sample; and
(c) identifying one or more biomarkers from a difference in the immune response from the first non-human animal to the immune response from the second non-human animal.

2. The method of claim 1 further comprising:
(d) administering to the first non-human animal the one or more identified biomarkers; and
(e) isolating one or more antibody-generating cells from the first non-human animal.

3. (canceled)

4. The method of claim 2, further comprising generating one or more hybridomas from the one or more antibody-generating cells.

5. (canceled)

6. The method of claim 4, further comprising isolating one or more antibodies from the one or more hybridomas.

7. (canceled)

8. (canceled)

9. The method of claim 2, further comprising generating a specificity profile for the one or more antibodies.

10. (canceled)

11. (canceled)

12. (canceled)

13. (canceled)

14. (canceled)

15. (canceled)

16. (canceled)

17. (canceled)

18. One or more isolated antibodies produced by the method of claim 6.

19. The one or more antibodies of claim 18, wherein the one or more antibodies comprises a plurality of antibodies, wherein 1% to 100% of the antibodies in the plurality are produced by the method of claim 6.

20. The one or more antibodies of claim 18, wherein the one or more antibodies comprises a plurality of antibodies, wherein each antibody of the plurality of antibodies has a binding affinity of at least 10⁻⁷ M (K_{D}) for a transcription factor.

21. The method of claim 1, wherein the administering comprises immunizing.

22. (canceled)

23. (canceled)

24. (canceled)

25. The method of claim 1, wherein the first biological sample, second biological sample, or both, is substantially depleted of a common serum protein before (a), wherein the depleted common serum protein comprises albumin or IgG.

26. (canceled)

27. (canceled)

28. The method of claim 1, wherein the first or second biological sample is from a virus, bacteria, mycoplasma, parasite, fungus, plant, or animal.

29. (canceled)

30. (canceled)

31. The method of claim 1, wherein the first or second biological sample is a tissue sample or bodily fluid.

32. (canceled)

33. (canceled)

34. The method of claim 1, wherein the first biological sample comprises a disease or a condition specific protein.

35. The method of claim 1, wherein the first biological sample is from a subject with a disease or condition.

36. The method of claim 35, wherein the second biological sample is from a subject without the disease or condition.

37. (canceled)

38. (canceled)

39. The method of claim 1, wherein the first biological sample is from a subject at one timepoint and the second biological sample is from the subject at a later timepoint.

40. The method of claim 1, wherein the first biological sample is from a subject before a treatment and the second biological sample is from a subject after the treatment.

41. The method of claim 1, wherein the first biological sample and the second biological sample are from the same species or from the same subject.

42. The method of claim 1, wherein the first biological sample and second biological sample are from different species or from different subjects.

43. (canceled)

44. (canceled)

45. (canceled)

46. The method of claim 1, wherein (b) comprises comparing serum samples from the first and second non-human animals.

47. (canceled)

48. The method of claim 1, wherein (b) comprises determining a level of the immune responses to one or more antigens.

49. The method of claim 1, wherein (b) comprises detecting binding of an antibody of the immune responses to one or more antigens.

50. The method of claim 48 or 49, wherein the one or more antigens are attached to an array.

51. (canceled)

52. (canceled)

53. (canceled)

54. The method of claim 48, wherein the array comprises a proteome array.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. The one or more antibodies of claim 18, wherein the one or more antibodies comprises a plurality of antibodies, wherein each antibody of the plurality of antibodies is monospecific.

67. (canceled)

68. The one or more antibodies of claim 18, wherein the one or more antibodies comprises a plurality of antibodies, wherein the plurality of antibodies comprises at least 50 different antibodies.

69. (canceled)

70. (canceled)

71. (canceled)

72. The one or more antibodies of claim 18, wherein the one or more antibodies comprises a plurality of antibodies, wherein, wherein each antibody of the plurality of antibodies is immobilized on a substrate.

73. The method of claim 6, further comprising validating the one or more antibodies by a method selected from the group comprising immunoprecipitation (IP), immunohistochemistry (IHC), Western Blot (WB), Enzyme Linked Immunosorbant Assay (ELISA), immunofluorescence (IF), immunocytochemistry (ICC), Chromatin Immunoprecipitation (ChIP), siRNA knockdown, or any combination thereof.

74. The method of claim 73, wherein the validation method is ChIP and wherein the one or more antibodies are validated for binding to a transcription factor.

75. The method of claim 74, wherein the transcription factor comprises a bound consensus DNA molecule, and wherein the validated one or more antibodies do not obstruct the binding of the transcription factor to the one or more consensus DNA molecules.

76. The method according to claim 74, wherein the transcription factor is further analyzed by ChIP-sequencing (ChIP-Seq).

77. (canceled)

78. (canceled)

79. (canceled)

80. (canceled)

81. (canceled)
